(19) United States
(12) Patent Application Publication
(10) Pub. No.: US 2026/0216234 A1
(43) Pub. Date: Jul. 30, 2026

(54) PHLOROTANNINS AND ALPHA-FUCANS TO TREAT TYPE 2 DIABETES MELLITUS

(71) Applicant: BIOATLANTIS LIMITED, Tralee (IE)

(72) Inventors: John T. O’SULLIVAN, Tralee (IE); Kieran J. GUINAN, Tralee (IE); Georgios TZORTZIS, Tralee (IE); Sivaprakash K. BALAKRISHNAN, Tralee (IE)

(73) Assignee: BIOATLANTIS LIMITED, Tralee (IE)

(21) Appl. No.: 19/113,237

(22) PCT Filed: Sep. 21, 2023
(86) PCT No.: PCT/EP2023/076156

§ 371 Date: Mar. 19, 2025

(30) Foreign Application Priority Data
Sep. 21, 2022 (EP) 22196976.9

(51) Int. Cl. A61K 31/737; A61K 31/085; A61K 31/357; A61K 36/03; A61K 36/04; A61K 45/06; A61P 3/10; A61P 5/50

(52) U.S. Cl. CPC A61K 31/737; A61K 31/085; A61K 31/357; A61K 36/03; A61K 36/04; A61K 45/06; A61P 3/10; A61P 5/50

(57) ABSTRACT

A composition comprising a phlorotannin or a α-fucan, or a combination thereof, for use in priming the endocrine system and enhancing insulin sensitivity, and for use in treating or preventing a disease or condition of an animal caused or facilitated by insulin insensitivity or insulin resistance such as obesity, high blood pressure, high cholesterol, T2DM, CVD, NAFLD, Metabolic Syndrome, kidney disease, Polycystic Ovary Syndrome, Depression, Alzheimer's, and/or for preventing or treating one or more of the symptoms of elevated blood glucose, hypertension, proinflammatory or inflammation state and prothrombotic state.

### FIELD OF THE INVENTION

The invention relates to a composition comprising one or more nutraceuticals for use in priming the endocrine system, for use in enhancing insulin sensitivity, and for use in treating and/or preventing insulin insensitivity, insulin resistance, chronic inflammation, and associated complications such as cardiovascular disease.

### CROSS REFERENCE TO RELATED APPLICATIONS

This application is a 35 U.S.C. § 371 National Phase Entry Application of International Application No. PCT/EP2023/076156, filed Sep. 21, 2023, which designates the U.S. and claims benefit under 35 U.S.C. § 119 (a) of European Patent Application No. 22196976.9 filed on Sep. 21, 2022, the contents of all of which are incorporated herein by reference in their entireties.

### SEQUENCE LISTING

The instant application contains a Sequence Listing which has been submitted electronically in XML format and is hereby incorporated by reference in its entirety. Said XML copy, created on Mar. 17, 2025, is named 048262-000123USPX_SL.xml and is 23,932 bytes in size.

### BACKGROUND TO THE INVENTION

According to the World Health Organization (WHO), the global incidence of type 2 diabetes mellitus (T2DM) in adults has more than doubled since 1980 to reach worldwide epidemic status. In 2014, it was estimated that 8.5% (422 million) of the human population had type 2 diabetes. Diabetic individuals are at increased risk of developing blindness, kidney failure, hypertension, heart attacks and strokes, which may reduce quality of life and lead to premature death. The WHO projects that diabetes will be the 7th leading cause of death by 2030. T2DM is largely associated with obesity, insufficient exercise and other lifestyle and genetic factors, typically beginning in middle or older age. However, because of our modern obesogenic lifestyle, substantially younger patients are increasingly diagnosed with this condition. Type 2 diabetes causes a ten year reduction in life-span.

T2DM, also known as Non-Insulin Dependent Diabetes Mellitus, or adult-onset diabetes, is a chronic metabolic disorder characterized by the body's inability to process glucose with insulin. Insulin, which is secreted by the pancreas, normally stimulates liver and muscle cells to take up glucose and store it as glycogen. However, in diabetic patients, they either do not produce enough insulin (type 1 diabetes) or produce insulin but are unable to process it due to an insulin resistance (type 2 diabetes) that eventually results in beta-cell exhaustion, leading to beta-cell destruction. When glucose continuously rises to dangerous levels in the bloodstream inducing metabolic overload and elevated protein glycation, blood vessels are damaged and vital organs are directly affected. Diabetic complications include the triopathy (neuropathy, retinopathy, and nephropathy) as well as limb ischemia, and hepatic, cardiovascular, and cerebrovascular diseases.

There has been significant and extensive research for effective long-term treatment for T2DM and many pharmaceutical drugs have been approved, including biguanides, alpha-glucosidase inhibitors, glucagon-like peptides, meglitinides, sodium glucose transporter 2 inhibitors, sulfonylureas, thiazolidinediones and gliptins. The objective of each is to maintain a blood glucose level range recommended by their physicians, typically with a fasting glucose level less than 100 mg/dl (5.6 mmol/L) (ADA, 2016).

Unfortunately, their efficacy in reducing the rate of progression of the disease may be quite low and these drugs can potentially produce side-effects which may reduce treatment efficacy and patient compliance. Moreover, those treatments are partial treatments, as their use does not resolve the spectrum of molecular and physiological abnormalities related to the diabetes processes. Alternative strategies used to treat diabetes focus on low-calorie diets and regular exercise, but these approaches are difficult to implement. Insulin treatment is required during the most advanced phases of the disease.

Prediabetes is the state in which a person has higher than normal blood glucose level, but not great enough to be diagnosed with diabetes. People who have these levels of glucose are at increased risk of developing T2DM and subsequent cardiovascular and other organ complications. At this stage the predominant abnormality is reduced insulin sensitivity. In reaction to increasing insulin resistance, beta-cells are forced to produce more insulin, or are triggered to proliferate and/or granulate, producing even more insulin. The overproduction of insulin or over activity of beta-cells can then lead to beta-cell exhaustion, leading to destruction of beta-cell populations. The pancreas can thus no longer provide adequate levels of insulin, resulting in elevated levels of glucose in the blood. Eventually, overt hyperglycemia and hyperlipidaemia occur, leading to the long-term complications associated with T2DM.

Management of insulin resistance in children and adults is essentially based on dietary and lifestyle changes, including healthier dietary habits and increased exercise. These practices can be very efficient in improving insulin sensitivity and in slowing the progression of the disease, but are very difficult to apply and therefore, are not followed by most patients, with their long-term efficacy being disappointing.

The focus of current T2DM treatment methods is to react to potentially immediate danger to one's life. Their shortcomings include hypoglycaemia, inconvenience and discomfort of administration for insulin injections, insufficiency to address severe hyperglycemia and treat the other dysmetabolic effects of T2DM for oral pharmaceutical drugs.

US 2008/280994 is directed to extracts of Ascophyllum, particularly Ascophyllum nodosum, and compositions comprising such extracts, and their medicinal use (useful in the treatment of Type 1 and Type 2 diabetes, or diabetes that is secondary to other conditions). The extracts they describe are alleged to comprise phlorotannins.

European Patent Publication No. EP2661181 is directed to a food product comprising a polyphenolic brown algae extract and at least one oil rich in omega-3 polyunsaturated fatty acids. The composition is used to treat and/or prevent chronic hyperglycemia on the retina, and also to have beneficial effects on insulin resistance, T2DM.

Japanese Patent Application No. 2013-129627 describes compositions comprising phlorotannins extracted from brown algae for the treatment of diabetes. However, human clinical trials have shown that extracts from species of brown seaweeds such as Ascophyllum nodosum and Fucus spp, (containing fucoidan and polyphenols such as phlorotannins) are observed to be largely ineffective or inconsistent in their effects, and are therefore unlikely to improve insulin sensitivity or metabolic markers, or have a role in glucose management or management of Metabolic Syndrome (MetS) at an individual or population level. (Keleszade et al., Molecules. 2021 Jan. 30; 26 (3): 714).

None of the currently available pharmacological treatments have been designed to prime the endocrine system or to maintain, improve, enhance or restore insulin sensitivity, the main driver of T2DM development and/or progression.

It is an object of the present invention to overcome at least one of the above-referenced problems.

### SUMMARY OF THE INVENTION

There is a significant need for a pharmacological composition and method that is directed towards treating the underlying T2DM disease process, and towards preserving and restoring the production and function of insulin and in particular insulin sensitivity which regulates blood glucose levels, and which is a major determinant of effective metabolic function. The invention described herein relates to a composition comprising a phlorotannin or an α-fucan, or a combination thereof, for use in priming the endocrine system; enhancing insulin sensitivity, treating or preventing insulin insensitivity and/or treating or preventing Type 2 Diabetes Mellitus (T2DM) in a subject; for treating inflammation in a subject; for boosting the immune system in a subject; for promoting recovery following exercise; for improving cognitive function and neuronal healing; relieving anxiety or depression in a subject; improving gut health; delaying aging; and treating autoimmune conditions.

The invention is summarised in the appended claims.

There is provided a composition comprising of an α-fucan and/or a phlorotannin at a ratio 1.5-3.0:1. Put another way, if the active of the composition consists of α-fucan alone, the composition consists of at least 60 mg α-fucan per 100 mg of the composition (at least 60% w/w α-fucan). If the active of the composition consists of phlorotannin alone, the composition consists of at least 40 mg of phlorotannin per 100 mg of the composition (at least 40% w/w phlorotannin). If the actives of the composition consist of the combination of α-fucan and phlorotannin, then a ratio of 1.5-3:1 of fucan:phlorotannin is present per 100 mg of the composition.

In one aspect, there is provided a composition for use in priming the endocrine system and achieving insulin sensitivity enhancement, endocrine homeostasis and/or treating or preventing endocrine dysregulation, deregulation, disruption or dysfunction.

In one aspect, there is provided a composition comprising at least one phlorotannin or at least one α-fucan, or a combination thereof, for use in treating or preventing insulin insensitivity and/or type 2 diabetes in a subject in need thereof.

In one aspect, there is provided a composition comprising at least one phlorotannin or at least one α-fucan, or a combination thereof, for use in priming the endocrine system, enhancing insulin sensitivity and treating or preventing insulin insensitivity and/or type 2 diabetes in a subject in need thereof.

In one aspect, the α-fucan and/or phlorotannin is isolated or derived from a marine or a terrestrial species of algae or plant. Preferably, the α-fucan and/or phlorotannin is isolated or derived from a micro- or macroalgae species belonging to the class of Phaeophyceae or Florideophyceae. Ideally, the α-fucan and/or phlorotannin is isolated or derived from a species selected from one or more of Laminariaceae, Fucaceae, Lessoniaceae, Alariaceae, Sargassaceae, Gigartinaceae, Ascophyllum, Laminaria, Alaria, Durvillea, Macrocystis, Chondrus, Ecklonia or any combinations thereof.

In one aspect, the phlorotannin and α-fucan are produced by means of synthetic chemistry, or a microbiological and/or biotechnology-related method, including growth of specific strains producing these compounds, organic synthesis techniques to construct these compounds or other GMO/non-GMO technologies.

In one aspect, the α-fucan and/or phlorotannin is isolated from a plant or other species. Preferably, the plant is selected from Aloe vera, Terminalia chebula, Perilla frutescens, Symplocos, Symphytum, Cactaceae, Zingiber zerumbet, Chrysanthemum morifolium, Tinospora cordifolia (guduchi), Nigella sativa, Gongronema latifolium, Pachira aquatic, Caesalpinioideae, Azadirachta indica, Artemisia dracunculus, Artemisia herbaalba, Andrographis paniculata L, Asphodelaceae, Mentha, Fabaceae, Achyranthes, Vachellia nilotica, Abelmoschus moschatus, Cinnamomum verum, Panax, Salvia officinalis, Tinospora cordifoli, Pterocarpus, Ocimum tenuiflorum, Momordica charantia, Mangifera indica, Syzygium cumini, Coccinia grandis, Caesalpinia bonduc, Liriope, Sarcopoterium, Swertia, Combretum, Gymnema sylvestre, Bauhinia, Ferula assafoetida, Carthamus tinctorius, Allium sativum, and Trigonella foenum-graecum.

In one aspect, the composition may be administered at regular intervals to an animal or human in the form of a powder, a gel, an emulsion or a liquid, a tablet, a capsule, a gel capsule, an injectable formulation, a food ingredient or supplement, or a nutraceutical ingredient or supplement.

In one aspect, the composition is suitable for administration in a daily dose of about 50 mg to about 1500 mg, that is, 50 mg, 55 mg, 60 mg, 65 mg, 70 mg, 75 mg, 80 mg, 85 mg, 90 mg, 95 mg, 100 mg, 150 mg, 200 mg, 250 mg, 300 mg, 350 mg, 400 mg, 450 mg, 500 mg, 550 mg, 600 mg, 700 mg, 750 mg, 800 mg, 850 mg, 900 mg, 950 mg, 1000 mg, 1050 mg, 1100 mg, 1150 mg, 1200 mg, 1250 mg, 1300 mg, 1350 mg, 1400 mg, 1450 mg, 1500 mg. Preferably, the composition is suitable for administration in a daily dose of about 60 mg, optionally at least about 120 mg per administration, at least about 160 mg per administration, at least about 180 mg per administration, at least about 200 mg per administration, or at least between about 200 and 320 mg per administration, greater than or equal to about 320 mg per administration, but not exceed 1500 mg per administration in a single dose. The daily dose can be administered as a single dose once per day, a single dose twice per day, a single dose three times a day, a single dose four times a day, a single dose five times a day, or a single dose up to 10 times a day provided that the daily dose is not exceeded. The daily administered dose of the composition can be from between 50 mg to 1500 mg.

In one aspect, the concentration of phlorotannin and α-fucan in the composition is at least about 40 mg and about 60 mg, respectively, on a dry weight basis.

In one aspect, when used in combination, the at least one phlorotannin and the α-fucan is present at a weight/weight ratio of between approximately 1:1 to 1:3. Preferably, when used in combination, the at least one phlorotannin and the α-fucan is present at a weight/weight ratio of between approximately 1:1.5 to 1:3.

In one aspect, the α-fucan is at least 15% sulphated and has a molecular weight of less than 10 KDa, or between 10 KD and 10,000 KDa, or more than 10,000 KDa. Preferably, the α-fucan is fucoidan.

In one aspect, the phlorotannin has a molecular weight of between 126 Da and 800 kDa, 126 Da to 300 kDa, 300 kDa to 600 kDa, or 600 kDa to 800 kDa

In one aspect, the phlorotannin is selected from one or more of a fucol, a fucophlorethol, a fuhalol, a carmalol, a phlorethol, an isofuhalol or an eckol, such as eckol and/or 7 phloroeckol.

In one aspect, the composition further comprises one or more selected from vitamins B1, B2, B3, B5, B6, B8, B9, B12, C, A, D, E, K1, and/or K2; obeticholic acid, corosolic acid, polyunsaturated fatty acids in the Omega 6 and/or Omega 3 family, orotic acid, pangamic acid, para-amino-benzoic acid, amygdalin, beta-glucans, carnitine, dimethylglycine, imeglimin, isoflavones, alginates, alginic acid, L-arginine, oxytocin, pectin, pyridoxamine, resveratrol, viniferine, and/or L-citrulline; stevia, sugar alcohols, sorbitol, isomalt, maltitol, mannitol, lactitol xylitol, hydrogenated starch hydrolysates, laminarin, plant extracts, probiotics, prebiotics, bioactives, oligo-elements, arsenic, boron, calcium, copper, iron, fluorine, iodine, lithium, manganese, magnesium, molybdenum, nickel, phosphorus, selenium, vanadium, and/or zinc; conjugated linolenic acid, lipoic acid carotenoids, carnitine, choline, Q10 coenzyme, phytosterols, polyphenols in the tannin and lignan family, and/or taurine; fructo-oligosaccharides, galacto-oligosaccharides; lactic ferments; yeasts; fungi; products derived from insects compatible with the food and pharmaceutical sector; marijuana and hashish; coating agents; aromas; acidifiers; anti-caking agents; thickeners; stabilizers; emulsifiers; filler agents; and/or excipients.

In one aspect, the composition is suitable for administration in a daily dose of about 50 mg to about 1500 mg.

In one aspect, there is provided a pharmaceutical composition comprising a phlorotannin or an α-fucan, or a combination thereof, and a pharmaceutically acceptable carrier.

In one aspect, the pharmaceutical composition is suitable for administration in a daily dose of about 50 mg to about 1500 mg.

In one aspect, there is provided a method for treating or preventing insulin insensitivity and/or type 2 diabetes in a subject in need thereof, the method comprising the step of administering an effective amount of the composition described herein.

In one aspect, there is provided a method for reducing blood glucose levels, for preventing or treating prediabetes or diabetes in a subject, the method comprising administering to the subject an effective amount of a composition described above. Preferably, the subject is obese and/or has one or more of the following characteristics: excess weight, glucose intolerance, prediabetes, metabolic syndrome and/or pathological high-blood pressure and/or the disorder is dyslipidemia, type 1 diabetes, type 2 diabetes, a non-alcoholic fatty liver disease, a cardiovascular pathology and/or a pathology related to insulin resistance.

In one aspect, there is provided a composition for use in a method for preventing or treating a disease or condition of an animal or human caused or facilitated by insulin insensitivity and/or insulin resistance such as obesity, high blood pressure, high cholesterol, T2DM, CVD, NAFLD, Metabolic Syndrome, kidney disease, Polycystic Ovary Syndrome, Depression, Alzheimer's, and/or for preventing or treating one or more of the symptoms of elevated blood glucose, hypertension, proinflammatory or inflammatory state and prothrombotic state.

In one aspect, a composition is provided for use as a method for priming or stimulating the endocrine system thereby enhancing insulin sensitivity, reducing insulin insensivity and/or reducing insulin resistance in a subject administered the composition.

In one aspect, a composition is provided for use as a method for priming or stimulating the endocrine system of a subject and/or modulating a range of processes at the biochemical, transcriptomic, physiological, immunological, metabolic, lipid and endocrine levels in a subject administered the composition.

In one aspect, a composition is provided for use as a method for priming or stimulating the endocrine system thereby maintaining, stimulating or modulating metabolic and endocrine homeostasis and/or treating or preventing endocrine dysregulation, deregulation, disruption or dysfunction in a subject administered the composition.

In one aspect, a composition is provided for use as a method for priming or stimulating the endocrine system of a subject by modulating a range of processes at the hormone metabolism, production, synthesis, secretion and gene expression levels in a subject administered the composition.

In one aspect, a composition is provided for use as a method for endocrine modulation and regulation of the endocrine system, hormonal status, insulin sensitivity, metabolism, immune function, and modulation and regulation of immune cells in infectious, autoimmune and immune mediated diseases in a subject administered the composition.

The composition described herein can be used as a free radical scavenger in the body of a subject who is administered the composition, and/or for preventing or reducing oxidative stress in a subject administered the composition.

In one aspect, there is a method for preventing or reducing oxidative stress in a subject, comprising administering to a subject in need thereof an effective amount of a composition described herein.

In one aspect, there is provided a method for preventing, reducing or scavenging free radicals in a subject, comprising administering to a subject in need thereof an effective amount of a composition described herein.

In one aspect, there is provided a method for preventing or treating chronic inflammation in a subject in need thereof, the method comprising administering the composition described above to the subject. In one aspect, the chronic inflammation is selected from tissue inflammation, systemic inflammation, pathogen induced inflammation, or chronic neurogenic inflammation and/or associated with an autoimmune disorder, a Th1-mediated inflammatory disease, SARS-COV 2 infection or other infectious agent or respiratory condition, or Alzheimer's disease.

In one aspect, there is provided a method for preventing or treating inflammatory, chronic or autoimmune or immune-mediated diseases, optionally selected from: Ulcerative colitis, irritable bowel syndrome, inflammatory bowel disease, colitis, Crohn's disease, psoriasis, rheumatoid arthritis, psoriatic arthritis, atopic dermatitis, Systemic Lupus Erythematosus (SLE), Multiple Sclerosis, Hashimoto's Thyroiditis, Graves'Disease, Celiac Disease, Graft-versus-Host Disease (GVHD), Asthma, and other such chronic conditions, autoimmune diseases, inflammatory diseases and immune-mediated diseases.

In one aspect, the composition is used in a method for priming or stimulating the immune system of a subject and/or modulating a range of processes at the transcriptomic, metabolic, lipid and endocrine levels.

In one aspect, there is provided a method for priming or stimulating the immune system of a subject, the method comprising administering to the subject an effective amount of the composition described herein.

In one aspect, a composition is provided for use as a method for reducing blood pressure, especially diastolic blood pressure of a subject in need thereof, the method comprising administering to the subject an effective amount of the composition described herein.

In one aspect, the method further modulates a range of processes at the transcriptomic, metabolic, lipid and endocrine levels.

In one aspect, the composition is used in a method for preventing or reducing exercise-related hyperlactatemia, muscular fatigue, muscular pain, cramp, weakness, and exhaustion and/or preventing impairment of physical performance, aerobic activity, anaerobic activity, exercise muscular endurance and skeletal muscle biogenesis.

In one aspect, the composition is used in a method for preventing, managing, reducing, or delaying signs of senescence, ageing and age-related conditions.

In one aspect, there is provided a method for preventing or reducing impairment of physical performance in a subject, the method comprising the step of administering to the subject an effective amount of the composition described herein.

In one aspect, impairment of physical performance is selected from exercise-related hyperlactatemia, muscular fatigue, muscular pain, cramp, muscular weakness, exhaustion, reduced aerobic activity, reduced anaerobic activity, reduced exercise muscular endurance, and reduced skeletal muscle biogenesis.

In one aspect, the composition is used in a method for preventing or treating a condition mediated by macrophage activation and/or nitric oxide production by macrophages.

The composition may have effects on the following parameters: blood pressure, low-density lipoprotein (LDL), high-density lipoprotein (HDL), blood lipids, triglycerides, blood glucose, fasting plasma glucose, fasting plasma insulin, HbA1c, insulin sensitivity (estimated by Homeostatic Model Assessment for Insulin Resistance (HOMA-IR) and HOMA-IR2), glycosylation of IgG, proinflammatory cytokines, anti-inflammatory cytokine, tumor necrosis factor alpha (TNFa), Interluekin-6 (IL-6), IL-8, IL-10, CD4+:CD8+ ratio, white blood cells counts and/or types, oxidative stress, MDA, Nitric Oxide, glutathione (GSH), Glutathione peroxidase (GPx), Superoxide dismutase (SOD), catalase (CAT), lactoperoxidase (LPO), pathogen load, gut microbiota composition, gut microbiota metabolic activity and/or the gut microbiome.

In one aspect, there is provided a method for maintaining and/or re-establishing central and peripheral nervous system health in a subject, the method comprising the step of administering to the subject an effective amount of the composition described herein.

In one aspect, the method supports neuronal repair and/or plasticity and/or post synaptic response, improves long term memory, improvise short term memory, learning and/or motor balance and coordination.

In one aspect, there is provided a method for improving emotional processing and preventing and/or relieving anxiety and/or emotional stress and/or depression in a subject, the method comprising administering to the subject an effective amount of the composition described herein.

In one aspect, there is provided a method for improving gut health, and/or promoting intestinal integrity and/or improving gut structure and function in a subject, the method comprising administering to the subject an effective amount of the composition described herein.

In one aspect, there is provided a method for preventing, managing, reducing or delaying signs of senescence, ageing and age-related conditions in a subject, the method comprising administering to the subject an effective amount of the composition described herein.

In one aspect, there is provided a method for preventing or treating a condition mediated by macrophage activation and/or nitric oxide production by macrophages in a subject, the method comprising administering to the subject an effective amount of the composition described herein.

In one aspect, there is provided a method for preventing or treating conditions, symptoms, complaints, or diseases mediated by endocrine system dysfunction and/or hormonal imbalances in subject, the method comprising administering to the subject an effective amount of the composition described herein.

In one aspect, the conditions, symptoms, complaints, or diseases mediated by endocrine system dysfunction and/or hormonal imbalances include those of a metabolic or estrogenic nature such as menopause.

In one aspect, the step of administering the effective amount of the composition comprises the administration of the composition one to three times daily, with the proviso that the daily dose of the composition is between 50 mg to 1500 mg. Preferably, the administration is in the form of local delivery, intravenous delivery, oral delivery, intranasal delivery, intramuscular delivery, intrathecal delivery, transdermal delivery, inhaled delivery, and topical delivery.

In one aspect, the administration is in the form of a medical food, a food, a food ingredient, a feed, a feed material, a feed ingredient, a supplement, or a nutraceutical ingredient or supplement.

Described herein is a composition consisting of a formulation of fucan and/or phlorotannin that has altering effects on one or more of: (I) free radicals scavenging, (II) oxidative stress, (III) low-grade and/or chronic inflammation, (IV) immune system function, (V) exercise-related hyperlactatemia, (VI) signs of senescence, (VI) nitric oxide production by macrophages, (VII) metabolic dysfunction, (VIII) fatigue, (IX) Long Covid symptoms such as but not limited to brain fog, and (X) infections; without any side effects in an easy to use natural way. In addition, clear effects on fasting plasma glucose, fasting plasma insulin, insulin sensitivity, HbA1c and prediabetic status have been demonstrated when a subject has been administered the claimed composition.

Accordingly in one aspect, the invention provides the use of a composition comprising fucans and/or phlorotannin in a method of improving insulin sensitivity, reducing insulin insensitivity, preventing or treating insulin insensitivity and reversing insulin resistance. In preferred embodiments, fucan and/or phlorotannin may be derived from more than one source which includes seaweeds and some echinoderms. The seaweed may be derived from the group which consists of Phaeophyceae, Florideophyceae, Laminariaceae, Fucaceae, Lessoniaceae, Alariaceae, Sargassaceae, Gigartinaceae, Ascophyllum, Laminaria, Alaria, Durvillea, Macrocystis, Chondrus, Ecklonia or any combinations thereof.

The brown macroalga of the class Phaeophyceae may be derived from one or more of the families Laminariaceae, Fucaceae, Lessoniaceae, Alariaceae, Sargassaceae, Ascophyllum, Laminaria, Alaria, Durvillea, Macrocystis, Ecklonia, or any combinations thereof. The red macroalga of the class Florideophyceae may be derived from one or more of the families Gigartinacea, Chondrus or combination thereof.

In one aspect, the at least one α-fucan and/or phlorotannin is isolated from a brown macroalga of the Ascophyllum species.

In one aspect, the at least one α-fucan and/or phlorotannin is isolated from a brown macroalga of the Laminaria species.

In one aspect, the at least one α-fucan and/or phlorotannin is isolated from a brown macroalga of the Sargassum species.

In one aspect, the at least one α-fucan and/or phlorotannin is derived from red alga, the red alga being Florideophyceae.

In one aspect, the α-fucan and/or phlorotannin may be derived by means of chemical synthesis and/or biotechnology approaches.

Methods of producing phlorotannins and/or α-fucans are disclosed in US2010056473A1, US2007003669A1, US2009270607A1, US2014328827A1, US2010048507A1, US2005196410A1, US2016074317A1, US2013338217A1, WO2006017943A1 and WO2005056035A1, and are incorporated herein by reference.

In one aspect, there is provided a supplementation regimen for enhancing insulin sensitivity, for preventing or treating insulin insensitivity or for preventing or treating insulin resistance in a subject by administering a composition comprising fucan and/or phlorotannin.

In one aspect, there is provided a supplementation regimen for reverting to a healthy status those individuals characterized as prediabetics.

In one aspect, there is provided a supplementation regimen for reducing blood glucose levels, preventing or treating prediabetes or diabetes in a subject by administering a composition comprising fucan and/or phlorotannin.

In one aspect, there is provided a supplementation regimen for scavenging free radicals and/or preventing or reducing oxidative stress in a subject by administering a composition comprising fucan and/or phlorotannin.

In one aspect, there is provided a supplementation regimen for preventing or treating chronic inflammation, wherein the chronic inflammation is tissue inflammation, systemic inflammation, pathogen induced inflammation, or chronic neurogenic inflammation and/or associated with an autoimmune disorder, a Th1-mediated inflammatory disease by administering a composition comprising fucan and/or phlorotannin.

In one aspect, there is provided a supplementation regimen for preventing or treating inflammatory, chronic or autoimmune or immune-mediated diseases, optionally selected from: Ulcerative colitis, irritable bowel syndrome, inflammatory bowel disease, colitis, Crohn's disease, psoriasis, rheumatoid arthritis, psoriatic arthritis, atopic dermatitis, Systemic Lupus Erythematosus (SLE), Multiple Sclerosis, Hashimoto's Thyroiditis, Graves' Disease, Celiac Disease, Graft-versus-Host Disease (GVHD), Asthma, and other such chronic conditions, autoimmune diseases, inflammatory diseases and immune-mediated diseases.

In one aspect, there is provided a supplementation regimen for preventing or treating inflammation caused by SARS-COV 2 infection or other infectious agent or respiratory condition by administering a composition comprising fucan and/or phlorotannin.

In one aspect, there is provided a supplementation regimen for treating a human previously infected with a virus and suffering with post viral symptoms, including but not limited to Long Covid, by administering a composition comprising fucan and/or phlorotannin.

In one aspect, there is provided a supplementation regimen for priming or stimulating the immune system of a subject and/or modulating a range of processes at the transcriptomic, metabolic, lipid and endocrine levels by administering a composition comprising fucan and/or phlorotannin.

In one aspect, a supplementation regime is provided for the purposes of priming or stimulating the endocrine system thereby enhancing insulin sensitivity and reducing insulin resistance by administering a composition comprising fucan and/or phlorotannin.

In one aspect, a supplementation regime is provided for the purposes of priming or stimulating the endocrine system of a subject and/or modulating a range of processes at the biochemical, transcriptomic, physiological, immunological, metabolic, lipid and endocrine levels by administering a composition comprising fucan and/or phlorotannin.

In one aspect, a supplementation regime is provided for the purposes of priming or stimulating the endocrine system thereby maintaining, stimulating or modulating metabolic and endocrine homeostasis and/or treating or preventing endocrine dysregulation, deregulation, disruption or dysfunction by administering a composition comprising fucan and/or phlorotannin.

In one aspect, a supplementation regime is provided for the purposes of priming or stimulating the endocrine system of a subject by modulating a range of processes at the hormone metabolism, production, synthesis, secretion and gene expression levels by administering a composition comprising fucan and/or phlorotannin.

In one aspect, a supplementation regime is provided for the purposes of endocrine modulation and regulation of biological parameters and processes such as the endocrine system, hormonal status, insulin sensitivity, metabolism, immune function, and modulation and regulation of immune cells in infectious, autoimmune and immune mediated diseases, by administering a composition comprising fucan and/or phlorotannin.

In one aspect, there is provided a supplementation regimen for preventing or reducing exercise-related hyperlactatemia, muscular fatigue, muscular pain, cramp, weakness and exhaustion and/or preventing impairment of physical performance, aerobic activity, anaerobic activity, exercise muscular endurance, skeletal muscle biogenesis by administering a composition comprising fucan and/or phlorotannin.

In one aspect, there is provided a supplementation regimen for preventing, managing, reducing or delaying signs of senescence, ageing and age-related conditions by administering a composition comprising fucan and/or phlorotannin.

In one aspect, there is provided a supplementation regimen for preventing or treating a condition mediated by macrophage activation and/or nitric oxide production by macrophages by administering a composition comprising fucan and/or phlorotannin.

In one aspect, there is provided a supplementation regimen for animals or humans that are susceptible to developing reduced insulin sensitivity, insulin insensitivity, insulin resistance, T2DM or obesity, including those susceptible due to genetic, environmental, dietary factors.

In one aspect, there is provided a supplementation regimen for animals or humans that are susceptible to developing to hyperglycemia, hyperlipidaemia, hyperinsulinemia, chronic inflammation, senescence, oxidative stress, prediabetes, hyperlactatemia and muscular fatigue or pain, endocrine system dysfunction, neurodegenerative conditions and/or disorders, emotional stress and/or hormonal imbalances, due to genetic, environmental, dietary factors.

The supplementation regimen for administration of the composition may be at least once daily or up to 3 times daily, with a dosage of at least 100 mg of the of α-fucan and phlorotannin per day. The supplementation regimen may be adapted to account for a personalised application.

The composition comprising fucan and/or phlorotannin described herein may be used in a method:
for preventing or treating insulin resistance,
for preventing or treating insulin insensitivity or reduced insulin sensitivity,
for improving insulin sensitivity,
for preventing or treating hyperglycaemia
for preventing or treating hyperlipidaemia,
for preventing or treating hyperinsulinemia,
for improving levels of HbA1c,
for preventing or treating chronic inflammation,
for priming the endocrine system,
for priming the endocrine function,
for inducing endocrine-mediated modulation of biological parameters, processes and systems,
for priming the immune system,
for reducing oxidative stress,
for delaying senescence,
for preventing or treating type 2 diabetes mellitus,
for preventing or treating prediabetes,
for regulating nitric oxide production by macrophages,
for preventing or treating hyperlactatemia and muscular fatigue or pain,
for reducing blood pressure,
for achieving the above in a safe, easy to use natural way.

The use of phlorotannins and/or α-fucan, singly or in combination, for priming the endocrine system, enhancing or improving insulin sensitivity or for use in preventing the development of conditions and diseases associated with insulin resistance is neither taught nor suggested by the prior art. Nor do they refer to the benefits of phlorotannins and/or alpha fucans, particularly those derived from seaweeds, to treat diseases and conditions caused by insulin insensitivity or reduced insulin sensitivity.

### Definitions

In the specification, the term “treating” (“treat”) is generally accepted to encompasses prohibiting, preventing, restraining, and slowing, stopping or reversing progression, severity, of a cause or resultant symptom of insulin insensitivity. The term includes causal or symptomatic treatment. As such, the methods of this invention encompass both therapeutic and prophylactic administration.

In the specification, the term “inflammation” should be understood to mean tissue inflammation, systemic inflammation, pathogen induced inflammation, or chronic neurogenic inflammation and/or associated with an autoimmune disorder, immune-mediated disease, a Th1-mediated inflammatory disease, SARS-COV 2 infection or other infectious agent or respiratory condition, or Alzheimer's disease.

In the specification, natural polysaccharides should be understood to comprise essentially sulfated alpha-L-fucose residues, and are known as fucoidans (or α-fucans). These are present in brown algae, some echinoderms and are the predominant polysaccharide in brown seaweeds, such as Ascophyllum nodosum and the Laminaria spp. A fucan is a sulphated polysaccharide, comprising at least two fucose saccharide monomers, wherein each monomer is linked to an adjacent monomer by a glycosidic bond. The polysaccharide molecule may be linear or branched. The fucan may be an α-fucan or a β-fucan. An α-fucan comprises at least one α-glycosidic bond. A glycosidic bond is where a carbon atom of a first monomer forms a bond, optionally a single order bond, with a carbon atom on an adjacent monomer. An α-glycosidic bond is where a functional group, optionally a hydroxyl group, is attached to a carbon atom of a first monomer and extends below the plane of the monomer (axially). Optionally, the C₁ carbon atom of a first monomer forms a bond, optionally a single order bond, with either the C₃ or C₄ carbon atom on an adjacent monomer. Optionally, the fucan is fucoidan.

Phlorotannins are natural-derived phenolic compounds. Phlorotannins are an oligomer or polymers of phloroglucinol produced only from micro- or macroalgae as a monomer and has various structures depending on how many types of monomers are combined. It is well known that such phlorotannins are suitable for human ingestion and have health promoting effects such as antioxidant effects, antibacterial effect, blood flow improvement effects, anti-hyperlipidaemic effects, and anti-inflammatory effects. These phlorotannin molecules have a very large number of hydroxyl groups capable of hydrogen bonding with the main ingredients of food, such as water, carbohydrates, and proteins.

In the specification, the term “Type 2 diabetes Mellitus (T2DM)” should be understood to mean a condition previously known as non-insulin-dependent diabetes mellitus (NIDDM), which develops as peripheral cells do not use insulin properly and then the pancreas loses its ability to produce enough insulin. Under current criteria, T2DM is diagnosed when fasting plasma glucose is >126 mg dL (7.0 mmol/L); or plasma glucose level is >200 mg/dl (11.1 mmol/L) at 2-hours post-glucose load of 75 g; or an HbA1c level >6.5%.

In the specification, the term “prediabetes” should be understood to mean a condition, also referred as impaired fasting glucose (IFG), impaired glucose tolerance (IGT), or a “prediabetic state”, where fasting plasma glucose is between 100 to 125 mg/dl (5.56-6.94 mmol/L); or plasma glucose level is between 140 to 19 mg/dl (7.78-11.06 mmol/L) at 2-hours post-glucose load of 75 g; or an HbA1c level between 5.7 and 6.4%. Prediabetes is a precursor condition to T2DM and without intervention and appropriate treatment, people with prediabetes are at risk for developing T2DM.

In the specification, the term “enhancing insulin sensitivity” should be understood to mean that the sensitivity to insulin increases for a condition in which muscle or cells are in a state in which they are unable to normally respond to endogenous and exogenous insulin (subjects, tissues and cells, etc.).

In the specification, the term “insulin sensitivity enhancer” should be understood to mean an agent capable of achieving the insulin sensitivity enhancement when administered to a subject.

In the specification, the term “insulin insensitivity” should be understood to mean the state at which insulin sensitivity is reduced but not to a level where it is classified as a state of insulin resistance; in this context, “insulin insensitivity” is classified using the Homeostatic Model Assessment for Insulin Resistance (HOMA-IR 2), where insulin insensitivity is defined as the state for HOMA-IR values 1.9-2.9, and whereby the ‘insulin sensitive’ state is represented by values of less than 1.9, with values less than 1.0 indicative of a state of homeostasis, whilst the ‘insulin resistant’ state exhibits values of greater than 2.9.

In the specification, the term “insulin resistance” should be understood to mean a disorder characterized by reduced sensitivity to muscle and cell insulin (i.e., a reduction in response to insulin). Insulin resistance is a component in the pathogenesis of multiple animal or human disease states and conditions including, but not limited to, metabolic syndrome, pre-diabetes, polycystic ovary syndrome, type 2 diabetes, dyslipidaemia, obesity, infertility, inflammatory disorders, cancer, inflammatory diseases, Alzheimer's disease, hypertension, atherosclerosis, cardiovascular disease and peripheral vascular disease.

In the specification, the term “priming the endocrine system” should be understood to mean a process of achieving insulin sensitivity enhancement, endocrine homeostasis and/or treating or preventing endocrine dysregulation, deregulation, disruption or dysfunction when administered to a subject.

In the specification, the term “endocrine modulation” should be understood to mean a process of inducing endocrine-mediated modulation and regulation of biological parameters, systems and processes such as the endocrine system, hormonal status, insulin sensitivity, metabolism, immunity, immune function, and modulation and regulation of immune cells in infectious, autoimmune and immune-mediated and metabolic diseases.

In the specification, the term “isolated” should be considered to mean material extracted from and/or removed from its original environment in which it naturally occurs, for example, in this instance a marine or terrestrial species of algae or plant. The removed material is typically extracted and/or purified separately from the environment in which it was located. Thus, the extracted and/or purified isolated phlorotannin and fucan in this instance ideally does not contain any significant amounts of impurities. In fact, the extracted and/or purified isolated phlorotannin and fucan contain impurities at such low levels that the levels are acceptable by regulatory authorities for human ingestion.

In the specification, the term “subject” should be understood to mean a “mammal” or “individual” and taken to mean an animal or human; however it should also include higher mammals for which the prophylaxis, therapy or use of the invention is practicable.

In this specification, the term “administering” should be taken to include any form of delivery that is capable of delivering the extracted, isolated or purified phlorotannin and/or fucan to a site of infection, including local delivery, intravenous delivery, oral delivery, intranasal delivery, intramuscular delivery, intrathecal delivery, transdermal delivery, inhaled delivery and topical delivery. Methods for achieving these means of delivery will be well known to those skilled in the art of drug delivery.

In this specification, the term “pharmaceutical composition” should be taken to mean compositions comprising a therapeutically effective amount of the claimed combination of phlorotannin and fucan, and a pharmaceutically acceptable carrier or diluent. In a specific embodiment, the term “pharmaceutically acceptable” means approved by an appropriate and relevant regulatory agency such as the US Food and Drug Administration (FDA), European Medicines Agency (EMA), European Food Safety Authority (EFSA) etc. or national or state authority or listed in the U.S. Pharmacopeia or other generally recognized pharmacopeia for use in animals, and more particularly in humans.

The term “carrier” refers to a diluent, adjuvant, excipient, or vehicle with which the combination of phlorotannin and fucan is administered. Such pharmaceutical carriers can be sterile liquids, such as water and oils, including those of petroleum, animal, vegetable, or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene glycol, water, ethanol and the like.

The composition, if desired, can also contain minor amounts of wetting or emulsifying agents, or pH buffering agents. These compositions can take the form of solutions, suspensions, emulsion, tablets, pills, capsules, powders, sustained-release formulations and the like.

The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in “Remington's Pharmaceutical Sciences” by E. W. Martin. Such compositions will contain a therapeutically effective amount of the therapeutic, preferably in extracted or purified form, together with a suitable amount of carrier so as to provide the form for proper administration to the patient. The formulation should suit the mode of administration.

In a preferred embodiment, the composition is formulated in accordance with routine procedures as a pharmaceutical composition adapted for intravenous administration to animals or human beings. Typically, compositions for intravenous administration are solutions in sterile isotonic aqueous buffer. Where necessary, the composition may also include a solubilizing agent and a local anaesthetic such as lignocaine to ease pain at the site of the injection. Generally, the ingredients are supplied either separately or mixed in a unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

“Effective amount” refers to the amount or dose of the composition, upon single or multiple dose administration to the patient, which provides the desired effect in the patient under treatment. An effective amount can be readily determined by the attending diagnostician, as one skilled in the art, by the use of known techniques and by observing results obtained under analogous circumstances. In determining the effective amount or dose of composition administered, a number of factors are considered by the attending diagnostician, including, but not limited to: the species of mammal; its size, age, and general health; the specific disease involved; the degree of or involvement or the severity of the disease; the response of the individual patient; the mode of administration; the bioavailability characteristics of the preparation administered; the dose regimen selected; the use of concomitant medication; and other relevant circumstances.

The term “food product” should be understood to include comestible products, such as foods and drinks. In particular, the food product is a dairy product, especially a fermented dairy product such as a yoghurt.

In the specification, the term “acceptable food additive” should be understood to mean a food supplement, a food enhancer (added to food items such as drinks and confectionary, etc.), vitamins, minerals, flavouring, colouring, and the like.

### BRIEF DESCRIPTION OF THE DRAWINGS

The invention will be more clearly understood from the following description of an embodiment thereof, given by way of example only, with reference to the accompanying drawings, in which:—

FIG. 1 is a graph which demonstrates the efficacy of the claimed composition in improving metabolic markers and enhancing insulin sensitivity in overweight adults at risk of developing T2DM following 4 wks supplementation. The effect of the composition (750 mg per capsule) (black) is presented as changes from baseline on metabolic markers, blood lipids and insulin sensitivity in overweight adults at risk of developing T2DM over a 4 wks supplementation period compared to placebo (grey). Supplementation with the composition resulted in significant reduction on fasting plasma glucose, fasting plasma insulin, and enhancement of insulin sensitivity. The effect on the insulin production was stronger than on the blood glucose, which suggests that the benefit of the claimed composition is not directly on blood glucose levels (e.g., a-glucosidase activity to reduce available glucose) but on improving insulin efficiency to deal with blood glucose. This is further supported by the significant enhancement in insulin sensitivity. No significant effect was recorded for blood lipids. *Denotes statistical significance at p<0.05; ** Denotes statistical significance at p<0.01. “Digestica” or “Digestica®” throughout the specification and figures denotes the composition of the claimed invention.

FIG. 2 is a graph which illustrates the impact of 4 weeks supplementation on the prediabetes/metabolic state of overweight adults. The overweight volunteers were characterised as prediabetics following the American Diabetes Association definition based on fasting plasma glucose (mmol/L) levels. Following 4 weeks supplementation the composition reverted the metabolic status of 3 out of the 4 prediabetic volunteers to normal, healthy levels (p<0.01). No change in the prediabetic status of any volunteers in the placebo group. * Denotes statistical significance at p<0.01. “Digestica” or “Digestica®” throughout the specification and figures denotes the composition of the claimed invention.

FIG. 3 is a graph which demonstrates the dose-response effect of the composition of the claimed invention (300 mg, 500 mg, 700 mg) on fasting plasma insulin, fasting plasma glucose, insulin sensitivity (HOMA IR) and HbA1c in overweight at risk of developing T2DM adults following 12 weeks supplementation of the claimed composition compared to berberine (positive control). The effect of treatments on insulin sensitivity (HOMA IR) was calculated as: HOMA (IR)=fasting insulin (microU/L)×fasting glucose (nmol/L)/22.5. “Digestica” or “Digestica®” throughout the specification and figures denotes the composition of the claimed invention. * Denotes statistical significance within treatment p<0.05. ^{a}denotes statistical significance between treatments p<0.05.

FIG. 4 is a graph which demonstrates the dose-response effect of the composition of the claimed invention (300 mg, 500 mg, 700 mg) on a panel of proinflammatory cytokines, (A) IL-1b, (B) IL-6, (C) TNFa, and (D) IL-10. “Digestica” or “Digestica®” throughout the specification and figures denotes the composition of the claimed invention. * Denotes statistical significance between treatments p<0.05. ** denotes statistical significance between treatments p<0.01. *** Denotes statistical significance within treatment p<0.001. Berberine is used as a positive control.

FIG. 5 illustrates the impact of the composition on the cerebellar genes' expression of alpha-synuclein in neonatal mice following maternal supplementation. Maternal supplementation with the claimed composition significantly improved the cerebellar expression of alpha-synuclein genes in the neonatal mice compared to the OXR1 phenotype (p<0.05). “Digestica” or “Digestica®” throughout the specification and figures denotes the composition of the claimed invention.

FIGS. 6A and 6B illustrates the expression of CD53 and c-fOS cerebellar genes in neonatal mice following maternal supplementation with the claimed composition. Maternal supplementation with the composition significantly improved the cerebellar expression of CD53 and c-FOS genes in the neonatal mice compared to the OXR1 phenotype (p<0.05). “Digestica” or “Digestica®” throughout the specification and figures denotes the composition of the claimed invention.

FIG. 7 illustrates the effect of different concentrations of α-fucan and/or phlorotannins in the composition of the claimed invention (different numbered Digestica® extracts in the bar charts) on cell viability and incretin hormone secretion in vitro. (A) Cell viability, (B) GLP-1 secretion, and (C) GIP secretion in STC-1 pGIP/Neo cells after 3 h exposure to Digestica® extracts at 5 mg/ml. Trypan blue was used to assess cell viability. Bars represent mean±SEM. Data analysed using one way ANOVA with Tukey's Multiple Comparisons post-test (*p<0.05, ** p<0.01, *** p<0.001 versus control; n=3). (D) illustrates high content analysis screening for the effects of Digestica® 4 on GLP-1 cellular content in vitro. Bar chart expresses GLP-1 immunofluorescence intensity per cell. Incubations are: HEPES buffer (control), Digestica® 4 at 1.25 mg/ml and Digestica® 4 at 2.5 mg/ml. Bars represent mean±SEM. Data analysed using one way ANOVA with Tukey's Multiple Comparisons post-test (*p<0.05, ** p<0.01, *** p<0.001 versus control; n=4). “Digestica®” throughout the specification and figures denotes the composition of the claimed invention.

### DETAILED DESCRIPTION OF THE DRAWINGS

### Examples

The examples below show the effects of the claimed compositions containing fucan and phlorotannin on healthy adults and overweight adults at risk of developing insulin resistance. The examples shown include human intervention studies using seaweed extract containing defined quantities of fucan and phlorotannin. Comparison with a commercially available, natural antidiabetic dietary supplement (Berberine, a quaternary ammonium salt from the protoberberine group of benzylisoquinoline alkaloids found in some plants like Berberis vulgaris (barberry), Berberis mexicana (tree turmeric), Mahonia aquifolium (Oregon grape), Hydrastis canadensis (goldenseal), Xanthorhiza simplicissima (yellowroot), Phellodendron amurense (Amur cork tree), Coptis chinensis (Chinese goldthread), Tinospora cordifolia, Argemone exicana (prickly poppy), and Eschscholzia californica (Californian poppy)) is also provided. An example presenting the cerebellar expression of genes associated with inflammation and insulin sensitivity following maternal supplementation in with the composition of fucan and phlorotannin in mice, is included as a model of the systemic effect of the composition.

### Example 1—Proof of Concept Study

A single site, randomized, double-blind, placebo-controlled clinical trial conducted at the University of Roehampton, London, UK, with twenty overweight adults randomized to take the claimed composition containing 160 mg of fucoidan and 70 mg of phlorotannins or placebo for 4 weeks, to study the effect of an α-fucan and a phlorotannin on metabolic markers and insulin sensitivity.

### Materials and Methods

Participants were eligible for the study if they met the following criteria:
18-65 years old.
BMI>25 kg/m².
No myocardial infarction/stroke or cancer in the past 12 months.
No diabetes (diagnosed or fasting blood glucose >7 mmol/l) or other endocrine disorders.
Not suffering from chronic coronary disease, kidney, bowel disease/gastrointestinal disorders.
Or not having a history of cholestatic liver disease or pancreatitis.
No medication for hyperlipidaemia, hypertension, inflammation, hypercoagulability, or medications affecting intestinal motility or absorption.
No history of alcohol/drug abuse.
Unplanned or ongoing weight loss program.
Not taking any dietary antioxidants or other phytochemical, prebiotic or probiotic supplements.
Not pregnant, breastfeeding, planning to become pregnant during the next 6 months or having fertility but not using effective contraception.
Did not take antibiotics in the previous 1 month.

Volunteers meeting the selection criteria were asked to participate in the selection process. During this process, fasting (12-hour) blood samples were taken and the BMI, waist circumference, and blood pressure were measured. Blood samples (~10 ml) were analysed for total cholesterol (TC), HDL cholesterol, triglycerides, glucose, and insulin at Affinity Lab (London, UK). Individuals with anaemia (Hb<14 g/dl (male), 11.5 g/dl (female)) or with ‘abnormal’ blood biochemistry (based on the above analysis) were automatically excluded. Measurements were used to identify individuals at higher metabolic risk and were subsequently invited to participate in the study.

For each of the two study groups (including stratum for gender), a 1:1 allocation ratio was used to randomly assign treatment A or B to each participant. Volunteers were asked to go to the university for a total of 2 visits. The study was a randomized, controlled, double-blind parallel trial using microcrystalline cellulose as a placebo. Volunteers were instructed to ingest the claimed composition and/or placebo daily for 4 weeks. The claimed composition and placebo were provided as powder capsules (750 mg in total; containing 230 mg of the composition, 15 mg Mannitol, 240 mg other carbohydrates, 40 mg protein, 225 mg inorganic matter (such as K₂O, sodium, calcium, zinc, iron, manganese, magnesium) and volunteers were instructed to ingest three capsules, one capsule before each meal, daily. The habitual diet was assessed through a pre-validated 4-day feeding log (2 weekends and 2 working days).

Capsule counting, at week 4, was used to assess the participant's compliance in taking the study product. A record of the date, the visit, and the amount of study product dispensed and returned was documented for each participant. Participants are defined as non-compliant if they have taken less than 80% of the study product.

On the 0^{th} and 4^{th} weeks of the intervention, volunteers visited the nutrition unit, sampled and measured.

At each visit, a fasting blood sample (~20 ml) was taken and used to analyze for:
Lipid profile (total lipids, low density lipoprotein (LDL) and HDL cholesterol, triglycerides, and non-esterified fatty acids).
Fasting plasma glucose and insulin.
Insulin sensitivity based on the ratio of glucose fasting measurements to insulin fasting measurements.

Statistical analysis was conducted using the intention-to-treat principle and missing data were imputed with baseline values for a conservative estimate (i.e., no change). Descriptive statistics are presented as mean±standard deviation. Outcomes were analysed using generalized linear models. The following covariates were added to the model: sex, age, and percentage weight change.

Ethical approval was granted by the University of Roehampton Ethics Committee. The study was carried out according to the Declaration of Helsinki, the National Health and Medical Research Council National Statement on Ethical Conduct in Research Involving Humans and the Notes for Guidance on Good Clinical Practice and the International Conference on Harmonisation Good Clinical Practise guidelines.

### Results

The demographic characteristics of the study population are shown in Table 1. There were no significant difference between the two treatment groups.

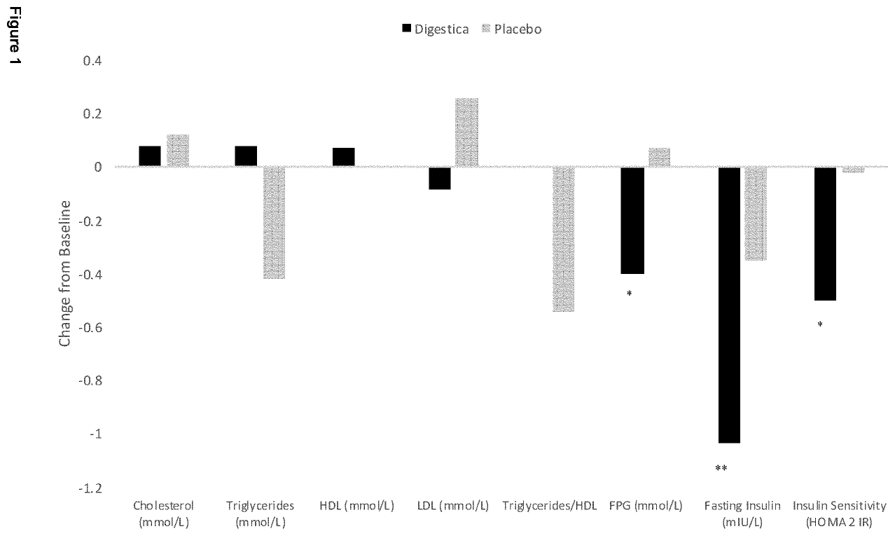

The daily intake of the claimed composition for 4 weeks resulted in significant improvements in fasting plasma glucose and insulin levels. No effect was detected on blood triglycerides, total cholesterol, and total cholesterol to HDL cholesterol ratio (FIG. 1).

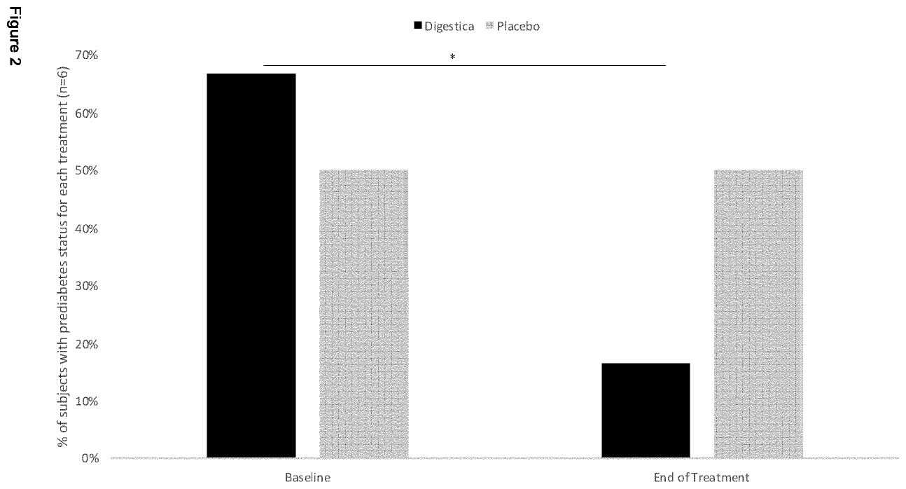

Additionally, in 75% of the people characterized as prediabetics based on the American Diabetes Association Guidelines, intake of the claimed composition resulted in them reverting to a healthy status (see FIG. 2).

TABLE 1
Baseline characteristics of subjects participating in the study.

| | Digestica | Placebo | p value |
|---|---|---|---|
| Age (yr) | 36.8 | 34.7 | 0.78 |
| Height (cm) | 164.2 | 168 | 0.41 |
| Weight (Kg) | 82.7 | 91 | 0.24 |
| BMI | 30.9 | 32 | 0.72 |
| Fat (%) | 40.6 | 41.6 | 0.87 |
| Fat Mass (Kg) | 34.5 | 38.8 | 0.56 |
| Free Fat Mass (Kg) | 51.8 | 52.2 | 0.95 |
| FPG (mmol/L) | 5.6 | 5.48 | 0.64 |
| Fasting Insulin (mIU/L) | 12.71 | 14.58 | 0.43 |
| Cholesterol (mmol/L) | 4.89 | 4.58 | 0.37 |
| Triglycerides (mmol/L) | 0.89 | 1.52 | 0.13 |
| HDL (mmol/L) | 1.29 | 1.12 | 0.5 |
| LDL (mmol/L) | 3.18 | 2.86 | 0.29 |
| Triglycerides/HDL | 0.75 | 1.6 | 0.13 |
| Insulin Sensitivity (HOMA 2 IR) | 3.23 | 3.52 | 0.59 |

### Conclusion

The claimed composition significantly improved insulin sensitivity and reduced insulin insensitivity and insulin resistance (p<0.05) as a result of a strong trend to reduce fasting plasma glucose and insulin in people with high BMI at risk of type 2 diabetes.

### Example 2—Dose Response Study

A single-centre, prospective, double-blind, randomised, placebo-controlled, parallel-group, dose response study consisting of 5 arms: a placebo control (Cellulose microcrystalline, Alfa Aesar), a positive control (400 mg Berberine per capsule, Thorne Research Berbercap), low dose of claimed composition (400 mg per capsule containing 81 mg fucoidan and 42 mg phlorotannins, 8 mg mannitol, 128 mg other carbohydrates, 21 mg protein, 120 mg inorganic matter), an intermediate dose of the claimed composition (500 mg per capsule, containing 101 mg of fucoidan and 52 mg of phlorotannins, 10 mg mannitol, 160 mg other carbohydrates, 27 mg protein 150 mg inorganic matter) and a high dose of the claimed composition (700 mg per capsule containing 141 mg of fucoidan and 73 mg of phlorotannins, 14 mg mannitol, 224 mg other carbohydrates, 37 mg protein, 210 mg inorganic matter). This study was conducted in the University of Roehampton Sport and Exercise Research Laboratory, Department of Life Sciences (Whitelands College, UK). The study consisted of two phases: a treatment period (4 weeks) and a wash-out period (4 weeks). Total of 50 participants were randomized to 5 treatments groups: placebo (n=10), 400 mg berberine (n=10), 400 mg of the claimed composition (n=10), 500 mg of the claimed composition (n=10) or 700 mg of the claimed composition (n=10). During the treatment period, participants were asked to take their assigned study product 3 times a day, each time 1 capsule, 30 minutes prior to having a meal (breakfast, lunch, and dinner).

During the washout period, participants were asked to maintain their typical diet and physical activity levels.

### Materials and Methods

Eligible participants had to meet the following criteria:

### Inclusion Criteria:

Females and males, aged 18 years to 65 years.
Body Mass Index (BMI) 27-35 kg/m2.
Not dieting within the last month and not having lost >5% body weight in the previous year.
No increased physical activity levels in the past 2-4 weeks or intending to modify them during the study.
Understands and is willing, able and likely to comply with all study procedures and restriction including being willing to follow the nutritional advice.
Able to eat most everyday foods.
Habitually consumes three standard meals a day (i.e., breakfast, lunch, and dinner).

### Exclusion Criteria:

Significant health problems (e.g., hypercholesterolaemia, diabetes, GI disorders).
Taking any medication or supplements known to affect mineral or glucose metabolism within the past month and/or during the study.
Pregnant, planning to become pregnant or breastfeeding.
History of anaphylaxis to food.
Known allergies or intolerance to foods and/or to the study materials (or closely related compounds) or any of their stated ingredients.
BMI <27 kg/m2 or >35 kg/m2.
Volunteers self-reporting currently dieting or having lost >5% body weight in the previous year.
Participants with abnormal eating behaviour.
Participation in another experimental study or receipt of an investigational drug/product within 30 days of the screening visit.
Volunteers who have significantly changed their physical activity in the past 2-4 weeks or who intend to change them during the study.
Participants receiving systemic or local treatment likely to interfere with the evaluation of the study parameters.
Participants on specific food avoidance diets.
Participants who work in appetite or feeding related areas.

An online random number generator (GraphPad QuickCalcs) was used to assign participants to treatment groups. This method ensured that information about group allocation remained concealed. When study visits and analysis of primary outcome were completed, the independent researcher provided the codes for un-blinding and treatment grouping.

All test products were encapsulated and blind packaged by BioAtlantis Ltd. (Tralee, Co. Kerry, Ireland). All study products had similar taste, appearance and were blended, encapsulated and blind packaged in blister packs, placed in opaque containers of identical colour.

The anthropometric variables monitored throughout the study included height, weight, body mass index, waist and hip circumference, and waist to hip ratio. For height and weight measurements, participants were requested to remove their socks, shoes, heavy garments, and heavy items prior to taking measurements. Height was measured to the nearest 0.1 cm using a portable stadiometer (Leicester Height Measure, Seca Ltd, Birmingham, United Kingdom) while the participants were standing erect with their feet together and shoulders level with the head oriented in the Frankfort horizontal plane. Body mass was measured in the fasted state to the nearest 100 g using a calibrated digital scale (Seca 707, Seca Corporation, Hamburg, Germany) placed on a hard, level surface. Body mass index was subsequently calculated by dividing body weight in kilograms by the square of the body height in meters (kg/m2). Waist and hip circumference were measured using an anthropometric tape (Seca 201, Hamburg, Germany) over light clothing to the nearest 0.1 cm while the subjects were in the standing position at the end of gentle expiration. The waist circumference was measured at the mid-point between the lowest rib margin and anterior superior iliac crest and hip circumference was measured at the maximum protuberance of the buttocks. Waist to hip ratio was subsequently calculated by dividing the waist circumference (in cm) by the hip circumference (in cm). All anthropometric measures were taken by the same investigator to ensure consistency.

Body composition was assessed by bioelectrical impedance analysis (BIA) using a Tanita BC-418 MA Segmental Body Composition Analyser, which incorporates eight tactile electrodes (Tanita Corporation, Tokyo, Japan). To ensure the predictive accuracy of these equations, each participant was asked to strictly comply with the following BIA pre-testing guidelines: no exercise within 12 hr of the test, no eating or drinking within 4 hr of the test, no alcohol consumption within 48 hr of the test, urination within 30 minutes of the test, no diuretic medications within 7 days of the test, and not to use lotion on hands or legs. Prior to measurement, participants were asked to take off all jewellery, shoes, socks, and heavy clothing and to empty their pockets. The device was calibrated to account for the weight of clothing (0.5 kg). Following subject age, height and gender input, weight was recorded automatically. The Tanita software uses inbuilt prediction equations for the body composition calculation taking into consideration subjects' activity level (category “standard” for a common individual or category “athlete” for an active individual). All study participants were classified as standard. The values of body weight, fat mass (FM), fat-free mass (FFM) and total body water (TBW) were reported in kg, except body fat that was reported in percentage.

The blood pressure was measured according to WHO guidelines using automated sphygmomanometer at the midpoint of the left arm after participants were rested for at least 5 minutes. Three blood pressure readings were taken for all participants with 1-minute intervals, then the mean value of these 3 measurements was set as the final blood pressure value.

For each volunteer, fasting blood samples were collected after 12 hr of overnight fast by a trained phlebotomist from the participant's antecubital vein using a 23G butterfly needle (Greiner Bio-One GmbH, Kremsmünster, Austria) into one 9 ml K2EDTA tube, one 9 ml Lithium Heparin tube, one 2 ml Sodium Fluoride/Potassium Oxalate tube and one 2 ml K2EDTA tube (Vacuette®; Greiner Bio-One GmbH, Kremsmünster, Austria). Following collection, all samples were kept on ice until centrifugation. Plasma samples were recovered by centrifugation at 1000 g for 15 minutes, dispensed into 1.5 ml microcentrifuge tubes and frozen at −20° C. within 1 hr from the collection.

Participants were asked to maintain their typical diet and physical activity levels throughout the study. At each visit, participants were given a food diary and asked to record all foods and beverages consumed over a 4-day period (including one weekend day). An electronic scale (Salter Arc Electronic Kitchen Scale) was provided along with photographic and written instructions. Upon collection, each food diary was checked for completeness by the same clinical nutritionist. All dietary analyses were carried out in Dietplan7 (Forestfield Software) using the McCance and Widdowson's composition of foods integrated data set unless specific food brands were provided in which case nutritional composition information was manually inputted as a user added food.

The physical activity levels of study participants were assessed using the General Practice Physical Activity Questionnaire (GPPAQ). The GPPAQ is a validated screening tool, used in primary care to assess the physical activity levels of adults (16-74 years) that was accredited by the Department of Health and developed by the London School of Hygiene and Tropical Medicine. Based on their responses, participants were classified into 4 categories; 1) Inactive (sedentary job and no physical exercise or cycling), 2) Moderately inactive (sedentary job and some but <1 hour physical exercise and/or cycling per week OR standing job and no physical exercise or cycling), 3) Moderately active (sedentary job and 1-2.9 hours physical exercise and/or cycling per week OR standing job and some but <1 hour physical exercise and/or cycling per week OR physical job and no physical exercise or cycling), 4) Active (sedentary job and ≥3 hours physical exercise and/or cycling per week OR standing job and 1-2.9 hours physical exercise and/or cycling per week OR physical job and some but <1 hour physical exercise and/or cycling per week OR heavy manual job).

All datasets were checked for normality using the D'Agostino-Pearson normality test. Non-normally distributed the data were transformed using the natural log and parametric tests were applied. Where data could not be normalised, non-parametric tests were applied, and data were reported as median (interquartile range (IQR)). Normally distributed data were reported as mean (SD). Differences between the treatment groups and the controls were compared using the two-way ANOVA or Kruskal-Wallis test for normally or non-normally distributed data, respectively followed by Tukey's post hoc test to determine where significant differences existed among the groups. All statistical analyses were performed using GraphPad Prism 9 (GraphPad Software, San Diego, California, USA), and a P≤0.05 was considered statistically significant.

The study was approved by the University of Roehampton Research Ethics Committee (Ethics reference number: LSC 18/247). All participants signed written informed consent after reading the participant information sheet and completed medical screening questionnaire prior to participating in the study.

### Results

The baseline characteristics of the study population are presented in Table 2. There were no statistically significant differences between the study arms.

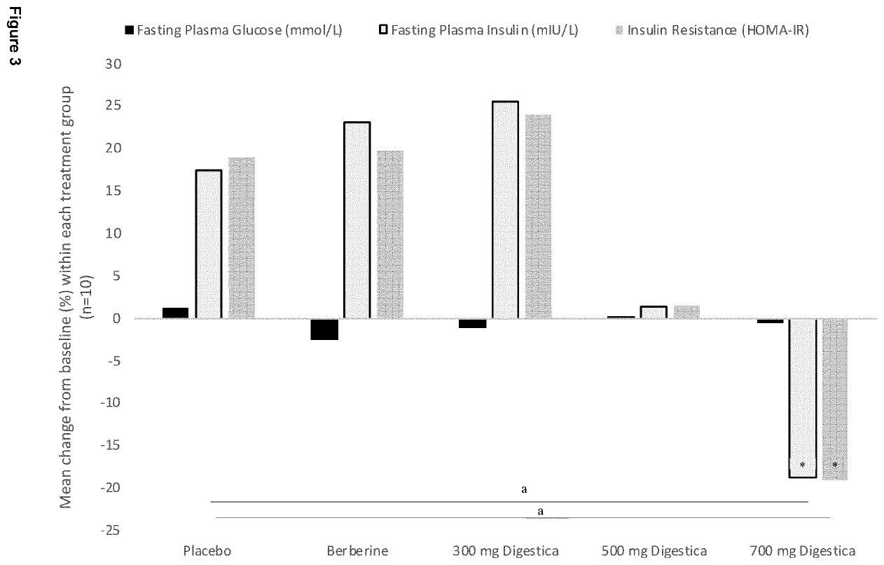

The impact of the treatments on metabolic function following 4 weeks of treatment is presented in FIG. 3. Daily intake of 700 mg of the claimed composition 3 times per daily, 30 minutes prior to having a meal has a significant effect on fasting plasma insulin (p<0.05) and insulin sensitivity (FIG. 3).

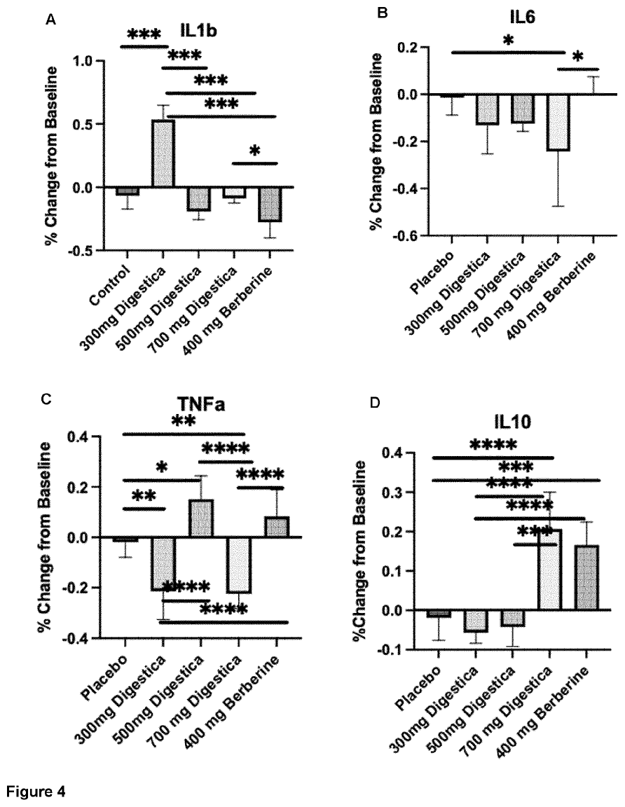

The impact of the treatments on markers of inflammation following 4 weeks of treatment is presented in FIG. 4. Overall, 700 mg Digestica three times per day, is shown to reduce the concentration of pro-inflammatory markers (decrease in IL-1b, IL-6, TNF-α) that are biomarkers of chronic inflammation, and increases the concentration of the anti-inflammatory IL-10 (FIG. 4).

TABLE 2
Baseline characteristics of the study population (Mean ± SD).

| | 700 mg | 500 mg | 300 mg | | 400 mg |
|---|---|---|---|---|---|
| | Digestica | Digestica | Digestica | Placebo | Berberine |
| | (n = 7) | (n = 6) | (n = 4) | (n = 6) | (n = 7) |
| Outcome | Mean ± SD | Mean ± SD | Mean ± SD | Mean ± SD | Mean ± SD |
| Weight (kg) | 68.6 ± 14.1 | 80 ± 27.2 | 82.3 ± 24.7 | 77.9 ± 14.6 | 71 ± 11 |
| Body mass | 25.3 ± 4.7 | 28.2 ± 8.3 | 26.5 ± 4.5 | 26.9 ± 4.8 | 25.6 ± 5.4 |
| index (kg/m²) | | | | | |
| Waist | 78.6 ± 11.5 | 86.4 ± 25.2 | 82 ± 13.1 | 80.1 ± 8.7 | 79.2 ± 7.3 |
| circumference | | | | | |
| (cm) | | | | | |
| Hip | 97 ± 8.8 | 104.9 ± 18.1 | 102.6 ± 10.4 | 104.3 ± 11 | 101 ± 8.9 |
| circumference | | | | | |
| (cm) | | | | | |
| Waist-hip ratio | 0.81 ± 0.08 | 0.81 ± 0.11 | 0.80 ± 0.07 | 0.77 ± 0.08 | 0.79 ± 0.04 |
| Body fat | 37.4 ± 7.9 | 38.8 ± 11.8 | 33.9 ± 6.7 | 36.9 ± 8.1 | 32.8 ± 14.7 |
| percentage (%) | | | | | |
| Fat mass (kg) | 26.5 ± 10.4 | 33.3 ± 18.4 | 28.2 ± 10.7 | 29.3 ± 9.7 | 23.6 ± 12.7 |
| Fat free mass | 42.1 ± 4.7 | 46.7 ± 11.2 | 54 ± 16.4 | 48.7 ± 9.1 | 47.4 ± 12.2 |
| (kg) | | | | | |
| Systolic BP | 125 ± 9 | 136 ± 21 | 119 ± 16 | 115 ± 9 | 117 ± 11 |
| (mm Hg) | | | | | |
| Systolic BP | 84 ± 8 | 83 ± 13 | 77 ± 9 | 73 ± 6 | 77 ± 10 |
| (mm Hg) | | | | | |

BP: blood pressure
Denotes statistical significance at p < 0.05

### Conclusion

Daily intake of 700 mg of the claimed composition, three times per day, significantly improves insulin sensitivity (p<0.05), and has the potential to revert insulin insensitivity and insulin resistance (from 2.05=prediabetes to 1.66=Healthy). Moreover, the impact of the claimed composition on the pro- and anti-inflammatory levels following the recommended daily intake has the potential to improve the health and quality of life of people suffering from immune-mediated and autoimmune conditions and diseases, and to treat and/or prevent those conditions and diseases.

### Example 3—Animal Model of Maternal Supplementation

Mutations in copper/zinc superoxide dismutase (SOD1) account for neurodegeneration in animal models. The OXR1 gene has been shown to extend the survival of mice expressing the mutant SOD1 gene, and mice lacking the OXR1 gene display extensive cerebellar neurodegeneration and die within 40 days of birth. Since previous studies have shown that the claimed composition has immune-modulatory properties, the claimed composition was tested in the OXR1 mice model. In this experiment, the effect of maternal feeding of the claimed composition on the expression of neonatal cerebellar genes (15 in total) that have been shown to change in the OXR1 model of neurodegeneration were examined. OXR1 heterozygous, pregnant mice (n=5) were administered the claimed composition or drinking water from conception through to suckling of newborn pups. Pups were culled at post-natal day 21, and the expression of cerebellar genes were analysed by QPCR at Oxford University, UK.

### Material and Methods

### Animals

All experiments were carried out with local ethical approval and a UK Home Office license granted under the Animals (Scientific Procedures) Act (1986). Female (28-30 g) and male (30-32 g) CD1 mice (Charles River, UK) were housed under normal laboratory conditions (12-h lightdark cycle, lights on at 7 a.m., 21±1° C., humidity 50±5%). The experimental unit was one mouse offspring, although litter effects were accounted for in the linear mixed-effects analysis. Group assignment and the order of testing and sample processing were randomized using the “Randomize range” function in Google Sheets. Experimental design and schematic diagrams were created with BioRender.com.

### Experimental Design

At E1, dams were randomly assigned to receive either standard drinking water provided by the animal facility (H₂O, five dams) or water supplemented with the claimed composition. Supplementation with the claimed composition continued through gestation and suckling. The composition contained 21.3% w/w Fucoidan and 9.3% w/w Phlorotannins on dry matter basis. Between P22-P27, pups received one intraperitoneal (I.P.) injection of saline or 1 mg/kg lipopolysaccharide (LPS). The open field test (OFT), light-dark box (LDB), and forced swim test (FST) were performed 24 hours later in that order. All tests were performed on the same day. After the FST, mice were culled, and the prefrontal cortex (PFC) and hippocampus were dissected from brains for qPCR.

### Quantitative Real Time PCR

Complementary DNA (cDNA) was synthesized with the RevertAid™ First Strand CDNA Synthesis Kit (Fermentas), using 1 μg of total RNA for each reaction. Quantitative RT-PCR reactions were subsequently carried out using SYBR Green PCR master mix (Applied Biosystems) with primers and cDNA added in optimized concentrations, using a StepOne real-time PCR machine (Applied Biosystems) with cycling conditions at 95° C. for 10 s, followed by 40 cycles of 55° C. for 15 s, 60° C. for 10 s. Gene amplification specificity was verified by melting curve analyses. The primer sequences used:

C1qa
SEQ ID NO. 1
5′-CAACGTGGTTATCTTTGACAAGGT-3′
and
SEQ ID NO: 2
5′-GAAGTTGAAGTAATAGAAGCCGGG-3′;
C1qb
SEQ ID NO. 3
5′-CACCAACGCGAACGAGAACT-3′
and
SEQ ID NO. 4
5′-GGCCAGGCACCTTGCA-3′;
C1qc
SEQ ID NO. 5
5′-CTACTTCGTCTACTACACATCGCA-3′
and
SEQ ID NO. 6
5′-CACCATGCCATTGTAGTCATTGAC-3′;
Cd52
SEQ ID NO. 7
5′-TCCTCCTCTTCCTCACTATCATTCT-3′
and
SEQ ID NO. 8
5′-GGCACATTAAGGTATTGGCAAAGA-3′;
Cd68
SEQ ID NO. 9
5′-CTACATCAGAGCCCGAGTAC-3′
and
SEQ ID NO. 10
5′-CTGGTAGGTTGATTGTCGTCTG-3′;
Ctss
SEQ ID NO. 11
5′-TACCAGGGTTCTTGTGGTGC-3′
and
SEQ ID NO. 12
5′-AGGGATATCAGCTTCCCCGT-3′;
Gapdh
SEQ ID NO. 13
5′-GCTACACTGAGGACCAGGTTGTC-3′
and
SEQ ID NO. 14
5′-AGCCCCGGCATCGAA-3′;
hSOD1
SEQ ID NO. 15
5′-GGCCAAAGGATHAAGAGAGGC-3′
and
SEQ ID NO. 16
5′-TGTGCGGCCAATGATGCAAT-3′;
Lyz2
SEQ ID NO. 17
5′-TCCTGACTCTGGGACTCCTC-3′
and
SEQ ID NO. 18
5′-AGCCAGCCATTCCATTCCTT-3′;
Mpeg1
SEQ ID NO. 19
5′-AGAAACCGGATCTACACAGTGAAA-3′
and
SEQ ID NO. 20
5′-GATTACGTGTGTGCCATAGTTGAG-3′;
Oxr1
SEQ ID NO. 21
5′-CAGTCGTGACTGGACAGGTTT-3′
and
SEQ ID NO. 22
5′-ATGGGCTACATCTGGAGTCG-3′;
Serpina3n
SEQ ID NO. 23
5′-CGAAACTGTACCCTCTGACTGTAT-3′
and
SEQ ID NO. 24
5′-TTGGCTATCTTGGCTATAAAGGGG-3′;
and
Vim
SEQ ID NO. 25
5′-CGGAAAGTGGAATCCTTGCAGG-3′
and
SEQ ID NO. 26
5′-AGCAGTGAGGTCAGGCTTGGAA-3′.

All values obtained were normalized with respect to mRNA levels of Gapdh.

### Statistical Analysis

Statistical tests were run in R 3.6.1 (R Foundation for Statistical Computing), and graphs were built in Prism 8.0.0 (Graphpad) and R. Grubbs' test for outliers was applied before statistical analysis, and statistically significant outliers were excluded. Group numbers in figure captions signify the final numbers after removal of outliers. Linear mixed-effects analysis was used to assess effects of maternal diet, LPS, and sex in offspring. Litter effects were accounted for in the analysis as a random effect. If two- or three-way interactions with sex were found, two two-way tests for males and females separately were performed to interpret sex-specific effects of maternal diet or LPS treatment. Otherwise, if no significant interactions of any variable with sex were found, male and female data were consolidated to minimize the number of comparisons and simplify the interpretation. Tukey tests were used for post hoc group comparisons when a significant interaction was detected. Orthogonal partial-least squares discriminant analysis (OPLS-DA) was used to identify differences in “metabolic fingerprints” between groups. Ten-fold external cross validation with repetition coupled with permutation testing was used to validate the models as previously described. The accuracy of the OPLS-DA models was determined on independent test data (10% of dataset) and compared to the models' performance in classifying randomly permuted data (accuracy≈50%). Specific metabolites driving differences in metabolic profiles were identified using average variable importance in projection (VIP) scores.

### Results

Of the 15 gene analysed, the expression of 3 in the neonatal mouse cerebellum were significantly influenced by maternal intake OF the claimed composition.

Alpha-synuclein (α-syn) is a protein with important normal function associated with its ability to interact with other molecules in a chaperone-like manner. α-syn is one of the most abundant proteins of the nervous system and can be found both centrally and in the peripheral nervous system. Although this protein is most often associated with a role in neurotransmission, a plethora of work has attributed a role of α-syn to disparate functions such as mitochondrial function, neurotransmitter production, and calcium homeostasis, amongst others. It is by far the most vetted pathogenic participant in the neurodegeneration that occurs in Parkinson's disease (PD) and other synucleinopathies such as multiple system atrophy (MSA).

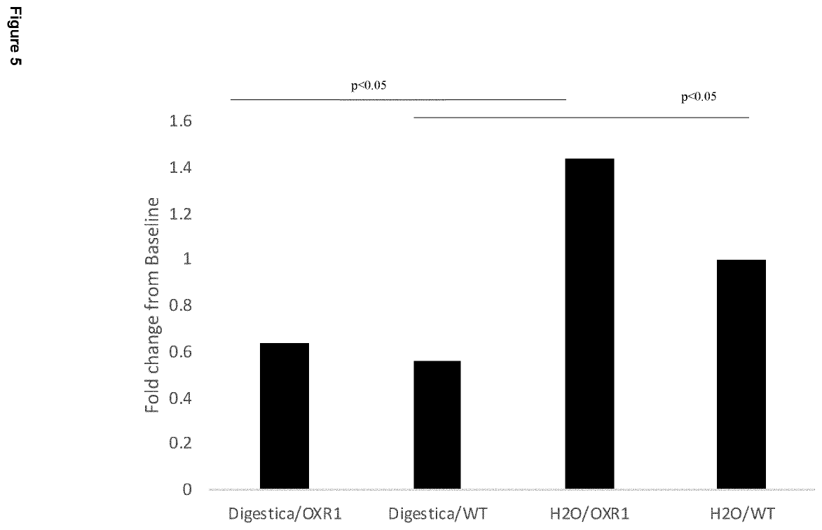

Over-expression of α-syn has been implicated in neurodegenerative disorders such as Parkinson's and Alzheimer's disease, and so its suppression in the mouse pup cerebellum by maternal feeding of the claimed composition suggest a reduction in neuronal death (FIG. 5). Another important function of α-syn is its expression in pancreatic beta cells where it interacts with Kir6.2 on insulin secretory granules, acting to downregulate insulin secretion, contributing thus to the development of T2DM.

The protein encoded by the CD53 gene is a member of the transmembrane 4 superfamily, also known as the tetraspanin family. Most of these members are cell-surface proteins that mediate signal transduction events and play a role in the regulation of cell development, activation, growth, and motility. This encoded protein is a cell surface glycoprotein that is known to complex with integrins. It contributes to the transduction of CD2-generated signals in T cells and natural killer cells and has been suggested to play a role in growth regulation. Familial deficiency of this gene has been linked to an immunodeficiency associated with recurrent infectious diseases caused by bacteria, fungi and viruses.

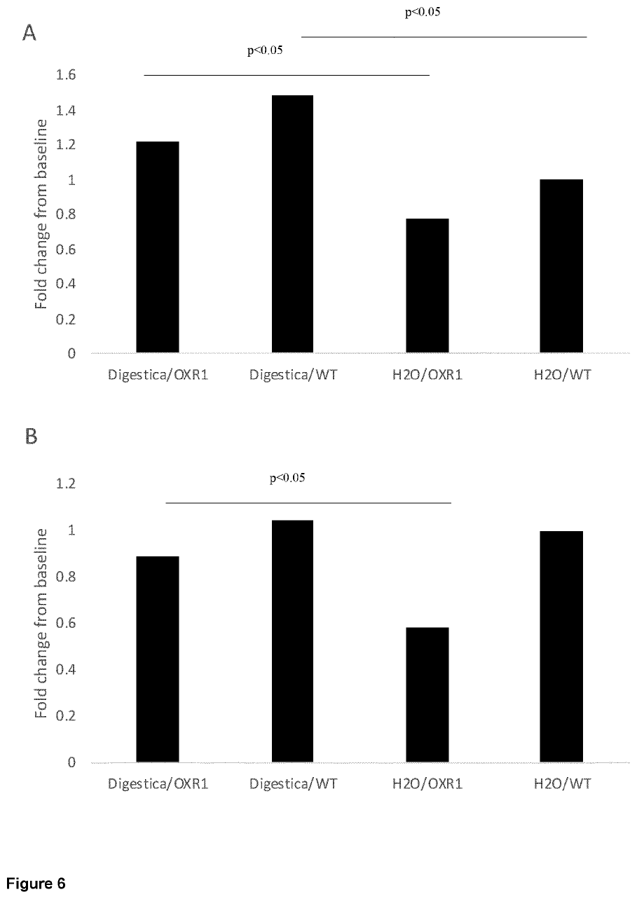

CD53 increased expression has been associated with neuroplasticity and neuroprotection. The normalization of CD53 levels in the mouse pup cerebellum by maternal feeding of the claimed composition (FIG. 6A) suggests that the intervention may reduce/impair neurodegeneration in ALS.

c-Fos is an immediate early response gene involved in cell proliferation and differentiation after extracellular stimuli. As an activator protein-1 (AP-1) heterodimer, c-Fos is considered a master switch that transduces short-term stimuli into long-term responses. c-Fos is currently used as a marker of neuronal activity and has been associated with several neural and behavioural responses to acute stimuli expression. In fact, age-related changes in neuronal function and plasticity with a concomitant decrease in the level of c-Fos have been observed in different brain regions. In addition to the above-mentioned alterations, the c-Fos−/− mice show behavioural alterations such as learning impairment, hyperactivity and abnormal sexual behaviour. Other research has shown that c-Fos plays an essential role in inducing B-cell proliferation regulating glucose stimulated insulin production.

cFos is an immediate-early gene that is often used as a marker of synaptic activity or strength. These results show that neurodegeneration in this ALS model maybe attenuated in the mouse pup cerebellum by maternal feeding of the claimed composition (FIG. 6B), since synaptic activity appears to be increased. In recently published studies, overexpression of c-Fos was reported to enhance glucose-stimulated insulin secretion in beta cells, while suppression of c-Fos expression impairs glucose stimulated insulin secretion in beta cells contributing towards the development of insulin resistance and T2DM.

### Conclusion

Maternal supplementation with the claimed composition resulted in the expression of cerebellar genes that regulate beta cells function and subsequent glucose stimulated insulin production.

### Example 4—In Vitro Incretin Hormones Production

Aim: To investigate how extracts derived by different methods and with different ratios of the claimed composition, give rise to different effects.

### Material and Methods

STC-1 cells transfected with a plasmid (pGIP/Neo) encoding neomycin phosphotransferase were utilized. These cells secrete both incretin hormones, GLP-1 and GIP. Cells were cultured in Dulbecco's Modified Eagle Medium (DMEM) containing 4.5 g/l D-glucose with L-glutamine, without sodium pyruvate (Gibco, Paisley, UK) and supplemented with 10% foetal bovine serum, 100 U/ml penicillin, 100 μg/ml streptomycin and 400 μg/ml geneticin (G418 disulfate salt; Sigma, UK). Cells were incubated in a 5% CO₂ humidified atmosphere at 37° C. and used between passage numbers 20-50 when 70-90% confluence had been reached.

STC-1 pGIP/Neo cells were seeded in 12 well plates at a density of 2 million cells per well with 1 ml DMEM and incubated overnight at 37° C. in a 5% CO₂ humidified atmosphere to allow attachment. Media was removed and cells were washed twice with HEPES buffer (pH 7.4) and pre-incubated in HEPES for 1 h. After removal of buffer, Digestica® samples 1 to 6, at 5 mg/ml (see Table 3 below), were reconstituted in HEPES and added to cells in triplicate for 3 h. After the incubation period supernatant was removed, centrifuged at 1000 g for 10 min to remove cellular debris and stored at −20° C. prior to analysis.

Trypan blue was used to assess cell viability. Trypsin (1 ml) was added to each well and incubated at 37° C. for 1 min to detach cells. Following this, 1 ml of DMEM was added to neutralise trypsin and resulting solution was centrifuged at 1000 g for 5 min. The supernatant was discarded and cells were re-suspended in DMEM. The cell suspension was added to trypan blue (1:1) and cell viability measured using a Countess Automated Cell Counter (Invitrogen, Life Technologies Ltd, UK).

For determining incretin hormone secretion from STC-1 pGIP/Neo cells, GLP-1 and GIP levels were measured by means of ELISA (Millipore, UK) in accordance to manufacturer's instructions. For total GLP-1 determination, assay buffer, standards and samples were incubated in the pre-coated 96 well plate overnight at 4° C. Plates were washed 5 times with wash buffer and detection conjugate was added to each well and incubated for a further 2 h. The plate was again washed 3 times prior to 20 min incubation with substrate. Stop solution was added to each well and the fluorescence measured on a fluorescence plate reader (Tecan Saffire II; Reading, UK) at excitation and emission wavelengths of 355 nm and 460 nm, respectively. For GIP analysis, standards and samples were incubated on a pre-coated plate for 1.5 h with agitation at room temperature. The plate was washed 3 times with wash buffer prior to addition of detection antibody and further incubated for 1 h with agitation. Enzyme solution was added for another 30 min after washing, followed by addition of substrate solution. After 20 min incubation, stop solution was added and absorbance measured at 450 nm (reference 590 nm) on a microplate reader (Tecan Saffire II; Reading, UK).

Test sample incubations for high content analysis (HCA) screening was performed as described above however using 96 well plates with 5×10⁴ cells per well. After the 3 h sample incubation period, pre-warmed fixing solution was added to each well for 10 min at room temperature. Plates were washed 4 times with PBS (pH 7.4) and permeabilization solution added for a further 30 min. Plates were again washed 4 times with PBS prior to a 1 h incubation with blocking solution (3% BSA in PBST). The blocking solution was removed before the addition of primary antibody (GLP-1 polyclonal antibody, 1:2500 dilution; produced in-house) and plates were incubated overnight at 4° C. The next day, plates were washed 3 times with PBS at 5 min intervals and FITC labelled secondary antibody (anti-rabbit IgG, 1:500 dilution; Abcam, Cambridge, UK) was added and incubated for 1 h at room temperature in the dark. Plates were again washed 3 times at 5 min intervals prior to addition of Hoechst staining solution. After 10 min incubation in the dark, staining solution was removed, plates were washed twice with PBS and read on an ArrayScan® HCS Reader (Thermo Scientific, UK).

All data are expressed as mean±SEM. GLP-1 and GIP concentrations were interpolated from standard curves and secretion was expressed per million cells per hour. Statistical analysis for in vitro studies was performed using one way ANOVA with Tukey's Multiple Comparison post-test to compare differences between groups (*p<0.05, ** p<0.01, *** p<0.001). All statistical analysis was performed using GraphPad Prism 5.0 software (GraphPad, California, USA).

TABLE 3
The Digestica ® 1-6 compositions used in the study are as follows:

| Composition | Seaweed species | Ratio (P:F) |
|---|---|---|
| Digestica ® 1 | Ascophyllum nodosum | 1:0.71 |
| Digestica ® 2 | Laminaria digitata | 1:10 |
| Digestica ® 3 | Ascophyllum nodosum | 1:4 |
| Digestica ® 4 | Ascophyllum nodosum | 1:2.26 |
| Digestica ® 5 | Laminaria digitata | 1:15 |
| Digestica ® 6 | Ascophyllum nodosum | 1:2.14 |

### Results

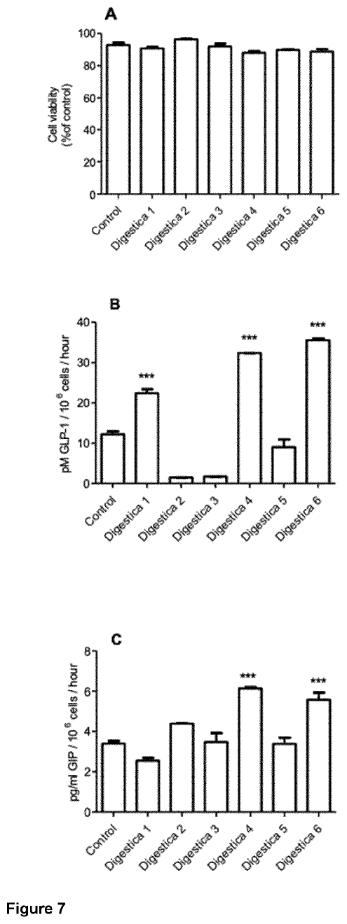

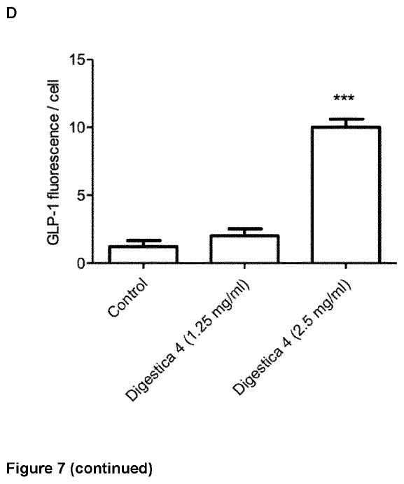

Digestica® compositions at concentrations of 5 mg/ml did not affect the viability of STC-1 pGIP/Neo cells as assessed by trypan blue. All compositions maintained cell viability of almost 100% (FIG. 7A).

Digestica® 1, Digestica® 4 and Digestica® 6 were able to increase GLP-1 secretion from STC-1 pGIP/Neo cells 1.8-fold, 2.7-fold and 2.9-fold, respectively (p<0.001), however Digestica® 2, Digestica® 3 and Digestica® 5 were ineffective at promoting GLP-1 secretion over the 3 h incubation period (p>0.05, FIG. 7B). Additionally, Digestica® 4 and Digestica® 6 were able to promote GIP secretion 1.8-fold and 1.6-fold, respectively (p<0.001) while the remaining four Digestica® extracts were unable to stimulate a response (p>0.05, FIG. 7C).

Digestica® 4 at 2.5 mg/ml produced a greater fluorescence intensity of GLP-1 within STC-1 pGIP/Neo cells when compared to control (p<0.001, FIG. 7D; green fluorescence indicates GLP-1, blue fluorescence indicates nuclei). Digestica® 4 at a lower concentration of 1.25 mg/ml did not significantly increase GLP-1 immunofluorescence over the 3 h incubation period (FIG. 7D).

### Conclusion

In vitro screening of various seaweed extracts derived by different extraction methods and containing the components of the claimed composition at different ratios, resulted in varying levels of incretin hormones secretion, suggesting differential impacts on insulin production and insulin sensitivity depending on the extraction process involved. The claimed composition resulted in the highest secretion of incretin hormones (GLP-1, GIP), potentially offering stronger stimulation of incretin hormone secretion, priming of the endocrine system and priming of endocrine function, as a way to regulate, and improve insulin production and improve insulin sensitivity.

### Example 5—Case Study on Hypertension

A 67-year-old male who has been diagnosed with type 2 diabetes and was treated with metformin for over 4 years, has for 2 months been ingesting 3 capsules per day of the claimed composition containing 160 mg of fucoidan (α-fucan) and 70 mg of phlorotannin. Prior to this period and for the preceding 2 years his systolic blood pressure was 144 mm Hg and his diastolic blood pressure was 99 mm Hg, which would classify him into the Primary Hypertension category. The patient had not been ingesting any medicines for high blood pressure.

Following the intake of the claimed composition, significant benefits on his blood pressure was recorded. The subject's systolic blood pressure was 129 mm Hg and the diastolic blood pressure was 89 mmHg, values what would be considered as healthy.

The subject also reported significant improvement in his mental health, mainly the level of stress he was feeling.

This report is not a controlled trial but does act as anecdotal evidence showing that the claimed composition may improve primary hypertension and restore the sufferers blood pressure levels and improve his quality of life.

### Example 6—Case Study on Muscular Cramps and Fatigue

A physically active male and former athlete has for 2 months been ingesting 3 capsules per day of the claimed composition containing 160 mg of fucoidan (α-fucan) and 70 mg of phlorotannin. The subject, aged 61, was in good health; however, he has experienced significant difficulties with muscle cramping after strenuous exercise, and long recovery time.

Following the intake of the claimed composition, the subject engaged in a strenuous physical activity, involving a 170 km cycle, which varied in terms of climb gradient and steepness. When comparing with his prior experience of undertaking strenuous long range cycles, the subject reported reductions in muscle cramping, fatigue and stress, following the intake of the claimed composition. He also reported shorter post-exercise recovery.

This report is not a controlled trial but does act as anecdotal evidence showing that the claimed composition may improve exercise performance and endurance, reduce cramping and stress and enhance post-exercise recovery.

Advantages of the method and composition of the invention include: it addresses insulin insensitivity, the main cause of prediabetes, reducing the risk of developing T2DM and not just the symptoms, e.g., blood glucose levels; it has extremely effective and strong insulin-sensitising and/or insulin sensitivity enhancing activity; it provides a means of priming the endocrine system and priming endocrine function; it addresses endocrine dysfunction, dysregulation, deregulation or disruption and lowers inflammation; it provides a means of improving insulin sensitivity; it is safer and easier to use as it is natural material rather than the synthetic pharmaceutical preparation as described above; its effectiveness in enhancing and/or improving insulin sensitivity in the person in need of, does not depend on dietary and lifestyle changes; it reduces C-reactive protein levels in the plasma, which are recognized to predict the future occurrence of diabetes and vascular disease; it preserves healthy cardiovascular function including blood vessels, by lowering LDL and/or increasing HDL level in the plasma; alpha-Fucan has been found to build and stimulate the immune system, reduces allergies, inhibits blood clotting, prevents ulcers, relieves stomach pains and detoxifies the body; α-fucans enhance phagocytosis helping the body fight bacteria and viruses such as herpes and SARS-Cov-2, and block the complement activation process that is believed to play an adverse role in chronic degenerative diseases such as Alzheimer's disease.

In the specification the terms “comprise, comprises, comprised and comprising” or any variation thereof and the terms “include, includes, included and including” or any variation thereof are considered to be totally interchangeable and they should all be afforded the widest possible interpretation and vice versa.

The invention is not limited to the embodiments hereinbefore described but may be varied in both construction and detail.

What is claimed is:

1. A composition comprising at least one phlorotannin and at least one α-fucan, or a combination thereof, for use in priming the endocrine system and enhancing insulin sensitivity, in a subject in need thereof.

2. (canceled)

3. (canceled)

4. The composition according to claim 1, wherein the at least one phlorotannin and α-fucan are isolated or derived from a marine or a terrestrial species of algae or plant.

5. The composition according to claim 1, wherein the at least one phlorotannin and α-fucan are isolated or derived from a marine or a terrestrial species of algae or plant, and wherein the algae is a microalgae or a macroalgae belonging to the class Phaeophyceae or Florideophyceae.

6. The composition according to claim 14, wherein the at least one phlorotannin and α-fucan are isolated or derived from a marine or a terrestrial species of algae or plant, and wherein the algae is selected from one or more of Laminariaceae, Fucaceae, Lessoniaceae, Alariaceae, Sargassaceae, Gigartinaceae, Ascophyllum, Laminaria, Alaria, Durvillea, Macrocystis, Chondrus, Ecklonia, or a combination thereof.

7. The composition of claim 1, wherein the phlorotannin and α-fucan are produced by means of synthetic chemistry, or a microbiological and/or biotechnology-related methodologies.

8. The composition according to claim 1, wherein the concentration of phlorotannin and α-fucan in the composition is at least about 40 mg and about 60 mg, respectively, on a dry weight basis.

9. The composition according to claim 1, wherein when used in combination, the at least one phlorotannin and the α-fucan is present at a weight/weight ratio of between approximately 1:1 to 1:3.

10. The composition according to claim 1, wherein when used in combination, the at least one phlorotannin and the α-fucan is present at a weight/weight ratio of between approximately 1:1.5 to 1:3.

11. The composition according to claim 1, wherein the α-fucan is at least 15% sulphated and has a molecular weight of less than 10 KDa, or between 10 KD and 10,000 KDa, or more than 10,000 KDa.

12. The composition according to claim 1, wherein the α-fucan is fucoidan.

13. The composition according to claim 1, wherein the phlorotannin has a molecular weight of between 126 Da and 800 kDa, 126 Da to 300 kDa, 300 kDa to 600 kDa, or 600 kDa to 800 kDa.

14. The composition according to claim 1, wherein the phlorotannin is selected from one or more of a fucol, a fucophlorethol, a fuhalol, a carmalol, a phlorethol, an isofuhalol, an eckol, and/or 7 phloroeckol.

15. The composition according to claim 1 further comprising one or more selected from vitamins B1, B2, B3, B5, B6, B8, B9, B12, C, A, D, E, K1, and/or K2; obeticholic acid, corosolic acid, polyunsaturated fatty acids in the Omega 6 and/or Omega 3 family, orotic acid, pangamic acid, para-amino-benzoic acid, amygdalin, beta-glucans, carnitine, dimethylglycine, imeglimin, isoflavones, alginates, alginic acid, L-arginine, oxytocin, pectin, pyridoxamine, resveratrol, viniferine, and/or L-citrulline; stevia, sugar alcohols, sorbitol, isomalt, maltitol, mannitol, lactitol xylitol, hydrogenated starch hydrolysates, laminarin, plant extracts, probiotics, prebiotics, bioactives, oligo-elements, arsenic, boron, calcium, copper, iron, fluorine, iodine, lithium, manganese, magnesium, molybdenum, nickel, phosphorus, selenium, vanadium, and/or zinc; conjugated linolenic acid, lipoic acid carotenoids, carnitine, choline, Q10 coenzyme, phytosterols, polyphenols in the tannin and lignan family, and/or taurine; fructo-oligosaccharides, galacto-oligosaccharides; lactic ferments; yeasts; fungi; products derived from insects compatible with the food and pharmaceutical sector; marijuana and hashish; coating agents; aromas; acidifiers; anti-caking agents; thickeners; stabilizers; emulsifiers; filler agents; and/or excipients.

16. The composition according to claim 1, wherein the composition is suitable for administration in a daily dose of about 50 mg to about 1500 mg.

17. A pharmaceutical composition method for use in treating or preventing insulin insensitivity, insulin resistance, and/or type 2 diabetes in a subject in need thereof, the method comprising administering an effective amount of a composition comprising a phlorotannin or an α-fucan, or a combination thereof, and a pharmaceutically acceptable carrier.

18. The pharmaceutical composition according to claim 17 for the use of method according to claim 17, wherein when used in combination, the at least one phlorotannin and the α-fucan is present at a weight/weight ratio of between approximately 1:1.5 to 1:3.

19. A composition for use in endocrine modulation to induce endocrine-mediated modulation and regulation of the endocrine system, hormonal status, insulin sensitivity, metabolism, immunity, immune function, and modulation and regulation of immune cells in infectious, autoimmune and immune-mediated diseases, the composition comprising at least one phlorotannin or at least one α-fucan, or a combination thereof.

20. The composition according to claim 19, wherein the at least one phlorotannin and α-fucan are isolated or derived from a marine or a terrestrial species of algae or plant.

21. The composition according to claim 19, wherein when used in combination, the at least one phlorotannin and the α-fucan is present at a weight/weight ratio of between approximately 1:1 to 1:3.


SEQUENCE LISTING (26 sequences)

SEQ ID NO: 1 - DNA, 24 bp, synthetic construct
  source 1..24: mol_type = other DNA
caacgtggttatctttgacaaggt

SEQ ID NO: 2 - DNA, 24 bp, synthetic construct
  source 1..24: mol_type = other DNA
gaagttgaagtaatagaagccggg

SEQ ID NO: 3 - DNA, 20 bp, synthetic construct
  source 1..20: mol_type = other DNA
caccaacgcgaacgagaact

SEQ ID NO: 4 - DNA, 16 bp, synthetic construct
  source 1..16: mol_type = other DNA
ggccaggcaccttgca

SEQ ID NO: 5 - DNA, 24 bp, synthetic construct
  source 1..24: mol_type = other DNA
ctacttcgtctactacacatcgca

SEQ ID NO: 6 - DNA, 24 bp, synthetic construct
  source 1..24: mol_type = other DNA
caccatgccattgtagtcattgac

SEQ ID NO: 7 - DNA, 25 bp, synthetic construct
  source 1..25: mol_type = other DNA
tcctcctcttcctcactatcattct

SEQ ID NO: 8 - DNA, 24 bp, synthetic construct
  source 1..24: mol_type = other DNA
ggcacattaaggtattggcaaaga

SEQ ID NO: 9 - DNA, 20 bp, synthetic construct
  source 1..20: mol_type = other DNA
ctacatcagagcccgagtac

SEQ ID NO: 10 - DNA, 22 bp, synthetic construct
  source 1..22: mol_type = other DNA
ctggtaggttgattgtcgtctg

SEQ ID NO: 11 - DNA, 20 bp, synthetic construct
  source 1..20: mol_type = other DNA
taccagggttcttgtggtgc

SEQ ID NO: 12 - DNA, 20 bp, synthetic construct
  source 1..20: mol_type = other DNA
agggatatcagcttccccgt

SEQ ID NO: 13 - DNA, 23 bp, synthetic construct
  source 1..23: mol_type = other DNA
gctacactgaggaccaggttgtc

SEQ ID NO: 14 - DNA, 15 bp, synthetic construct
  source 1..15: mol_type = other DNA
agccccggcatcgaa

SEQ ID NO: 15 - DNA, 21 bp, synthetic construct
  source 1..21: mol_type = other DNA
ggccaaaggathaagagaggc

SEQ ID NO: 16 - DNA, 20 bp, synthetic construct
  source 1..20: mol_type = other DNA
tgtgcggccaatgatgcaat

SEQ ID NO: 17 - DNA, 20 bp, synthetic construct
  source 1..20: mol_type = other DNA
tcctgactctgggactcctc

SEQ ID NO: 18 - DNA, 20 bp, synthetic construct
  source 1..20: mol_type = other DNA
agccagccattccattcctt

SEQ ID NO: 19 - DNA, 24 bp, synthetic construct
  source 1..24: mol_type = other DNA
agaaaccggatctacacagtgaaa

SEQ ID NO: 20 - DNA, 24 bp, synthetic construct
  source 1..24: mol_type = other DNA
gattacgtgtgtgccatagttgag

SEQ ID NO: 21 - DNA, 21 bp, synthetic construct
  source 1..21: mol_type = other DNA
cagtcgtgactggacaggttt

SEQ ID NO: 22 - DNA, 20 bp, synthetic construct
  source 1..20: mol_type = other DNA
atgggctacatctggagtcg

SEQ ID NO: 23 - DNA, 24 bp, synthetic construct
  source 1..24: mol_type = other DNA
cgaaactgtaccctctgactgtat

SEQ ID NO: 24 - DNA, 24 bp, synthetic construct
  source 1..24: mol_type = other DNA
ttggctatcttggctataaagggg

SEQ ID NO: 25 - DNA, 22 bp, synthetic construct
  source 1..22: mol_type = other DNA
cggaaagtggaatccttgcagg

SEQ ID NO: 26 - DNA, 22 bp, synthetic construct
  source 1..22: mol_type = other DNA
agcagtgaggtcaggcttggaa